SEQUENCE LISTING (32 sequences)

SEQ ID NO: 1 - DNA, 24 bp, Artificial Sequence
  synthetic sequence
tggacgcagacgatgtctttacca

SEQ ID NO: 2 - DNA, 24 bp, Artificial Sequence
  synthetic sequence
acatggccatgcctgggtctgaga

SEQ ID NO: 3 - DNA, 18 bp, Artificial Sequence
  synthetic sequence
caaatagccctggcagat

SEQ ID NO: 4 - DNA, 20 bp, Artificial Sequence
  synthetic sequence
tgatacaagggacatcttcc

SEQ ID NO: 5 - DNA, 21 bp, Artificial Sequence
  synthetic sequence
gaccacctttttagcctcctg

SEQ ID NO: 6 - DNA, 21 bp, Artificial Sequence
  synthetic sequence
agggaagggacttaggaatga

SEQ ID NO: 7 - DNA, 20 bp, Artificial Sequence
  synthetic sequence
cagatggcaccctccgatac

SEQ ID NO: 8 - DNA, 20 bp, Artificial Sequence
  synthetic sequence
gacacacctcggaagcagaa

SEQ ID NO: 9 - DNA, 17 bp, Artificial Sequence
  synthetic sequence
ccgccaaccgcaagatc

SEQ ID NO: 10 - DNA, 18 bp, Artificial Sequence
  synthetic sequence
accgacccaccgaagaca

SEQ ID NO: 11 - DNA, 20 bp, Artificial Sequence
  synthetic sequence
actgtgaaggcgcagaatca

SEQ ID NO: 12 - DNA, 20 bp, Artificial Sequence
  synthetic sequence
tgcctctatccagtcgacca

SEQ ID NO: 13 - DNA, 20 bp, Artificial Sequence
  synthetic sequence
ctcccttcttccctcccctc

SEQ ID NO: 14 - DNA, 20 bp, Artificial Sequence
  synthetic sequence
atctcgattacgggacacgc

SEQ ID NO: 15 - DNA, 20 bp, Artificial Sequence
  synthetic sequence
gccaagttccaaagtgagcg

SEQ ID NO: 16 - DNA, 20 bp, Artificial Sequence
  synthetic sequence
ctaccaggtcccttagggct

SEQ ID NO: 17 - DNA, 20 bp, Artificial Sequence
  synthetic sequence
ctcacgggtacagtaacgca

SEQ ID NO: 18 - DNA, 20 bp, Artificial Sequence
  synthetic sequence
ccacaaaagagccaaagccc

SEQ ID NO: 19 - DNA, 20 bp, Artificial Sequence
  synthetic sequence
ttatgcagcagagccccatc

SEQ ID NO: 20 - DNA, 20 bp, Artificial Sequence
  synthetic sequence
caagacggaagtcacgggaa

SEQ ID NO: 21 - DNA, 22 bp, Artificial Sequence
  synthetic sequence
aatgtgtccgtcgtggatctga

SEQ ID NO: 22 - DNA, 22 bp, Artificial Sequence
  synthetic sequence
agtgtagcccaagatgcccttc

SEQ ID NO: 23 - DNA, 18 bp, Artificial Sequence
  synthetic sequence
tgtgctgttccaacctct

SEQ ID NO: 24 - DNA, 18 bp, Artificial Sequence
  synthetic sequence
ttcctgaagaacaccctg

SEQ ID NO: 25 - DNA, 18 bp, Artificial Sequence
  synthetic sequence
ttgaaacgaaccctgaaa

SEQ ID NO: 26 - DNA, 18 bp, Artificial Sequence
  synthetic sequence
ttccctagccttccttct

SEQ ID NO: 27 - DNA, 20 bp, Artificial Sequence
  synthetic sequence
attttggtgtaagttctatg

SEQ ID NO: 28 - DNA, 25 bp, Artificial Sequence
  synthetic sequence
gactatatttctattgctgttgtga

SEQ ID NO: 29 - DNA, 18 bp, Artificial Sequence
  synthetic sequence
tcagcgttacaagaccaa

SEQ ID NO: 30 - DNA, 18 bp, Artificial Sequence
  synthetic sequence
tccaggtagcctctgttt

SEQ ID NO: 31 - DNA, 22 bp, Artificial Sequence
  synthetic sequence
aatgtgtccgtcgtggatctga

SEQ ID NO: 32 - DNA, 22 bp, Artificial Sequence
  synthetic sequence
agtgtagcccaagatgcccttc

(19) United States
(12) Patent Application Publication
(10) Pub. No.: US 2021/0205638 A1
(43) Pub. Date: Jul. 8, 2021

(54) PARATHYROID HORMONE AND REGENERATION OF DEGENERATIVE DISCS DISEASE

(71) Applicant: THE JOHNS HOPKINS UNIVERSITY, Baltimore, MD (US)

(72) Inventor: Xu CAO, Ellicott City, MD (US)

(21) Appl. No.: 17/191,419

(22) Filed: Mar. 3, 2021

Related U.S. Application Data
(63) Continuation of application No. 16/482,897, filed on Aug. 1, 2019, now abandoned.
(60) Provisional application No. 62/453,044, filed on Feb. 1, 2017.

(51) Int. Cl. A61P 19/10; A61P 35/00; A61K 38/29

(52) U.S. Cl. CPC A61P 19/10; A61K 9/0019; A61K 38/29; A61P 35/00

(57) ABSTRACT

The present invention provides methods for treatment of IVD degeneration and/or LDD in a subject having symptoms of IVD degeneration and/or LDD comprising administering to the subject an effective amount of Parathyroid hormone (PTH) or a functional fragment or analog thereof. Reduction in IVD degeneration and regeneration of IVD using the inventive methods is also provided.

### REFERENCE TO RELATED APPLICATIONS

This application is a continuation of U.S. patent application Ser. No. 16/482,897, filed Aug. 1, 2019, which is a 371 National Entry of PCT/US2018/016429, filed Feb. 1, 2018 which claims the benefit of U.S. Provisional Patent Application No. 62/453,044, filed on Feb. 1, 2017, which are hereby incorporated by reference in their entireties.

### STATEMENT OF GOVERNMENTAL INTEREST

This invention was made with government support under grant no. AR 063943, awarded by the National Institutes of Health. The government has certain rights in the invention.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Mar. 3, 2021, is named 37907-303_ST25.txt and is 6,378 bytes in size.

### BACKGROUND OF THE INVENTION

Lumbar disc disease (LDD) also known as intervertebral disc (IVD) degeneration, or degenerative disc disease or degenerative disc disorder (DDD), has long been documented as a disease of aging and is one of the most common musculoskeletal disorders. Degeneration has been detected as early as in teenage years, and severe degeneration is detected in 60% of 70-year olds. The numbers of people with associated lower back pain is at epidemic proportions worldwide, affecting up to 80% of individuals during their lifetime, and is the leading cause of disability. LDD imposes an enormous socio-economic burden, over $100 billion annually in the United States alone, and costs more than the combined costs of stroke, respiratory infection, diabetes, coronary artery disease and rheumatoid disease.

Despite the prevalence and significance of LDD, little is understood of its pathophysiology and there is no effective disease-modifying treatment. The intervertebral disc (IVD) can be separated macroscopically into two components; an outer network of collagen fibers, termed the annulus fibrosus (AF) which surrounds a hydrated, centrally located nucleus pulposus (NP). The NP is made up cells of notochordal origin surrounded by abundant proteoglycans, mainly aggrecan and collagen, which form the extracellular matrix (ECM). The NP functions to absorb axial compressive forces transmitted along the spine. The ECM is an essential element supporting NP cells for disc homeostasis. Proteoglycans with their water binding ability, provides resilience to compression whilst the collagen imparts tensile strength.

Individuals with mutations in ECM protein type II collagen have severe IVD degeneration. Genetic studies have reported associations between LDD and the genes encoding components of the ECM, specifically type IX collagen, aggrecan, and cartilage intermediate layer protein (CILP). CILP directly binds to TGF-β1 to inhibit TGF-β1-induced cartilage ECM protein synthesis. NP cells secrete TGF-β, which induces transcription of connective tissue growth factor (CTGF/CCN2) to increase extracellular matrix synthesis in the cartilage and discs. As such, TGF-β activity is critical for disc extracellular protein expression and function.

Parathyroid hormone (PTH), an FDA-approved anabolic therapy for osteoporosis, stimulates bone remodeling. PTH classically binds to the type 1 PTH receptor (PTH1R) and stimulates adenylate cyclase for formation of cyclic adenosine monophosphate (cAMP). Increased cAMP levels activate protein kinase A (PKA) to induce phosphorylation of cAMP response binding protein (CREB) a transcription factor involved in PTH mediated gene expression. PTH also regulates skeletal homeostasis by orchestrating signaling of local factors, including TGF-β via alternate pathways. Despite the well documented involvement of PTH in skeletal tissue homeostasis and calcium metabolism there is paucity of data regarding its role in disc degeneration.

Despite the prevalence and significance of DDD, thus far, there is currently no effective treatment to arrest the progression of degeneration or restore the disc function. Symptomatic relief of pain and surgeries such as golden standard spinal fusion are the widely adopted treatments for DDD nowadays, despite several drawbacks and obstacles of these treatment strategies. Many of the methods used to mitigate symptoms are either obscure or actually destructive to disc integrity. Even though tons of efforts have been made in recent years by injecting cells or biomaterials into the disc space, as well as replacement of bioengineered disc, none of which allowed truly backbone rebuilt, nor did these studies unveiled the mechanism underlying the homeostasis and rejuvenation of the disc tissues.

Thus, there still exists and unmet need for development of more effective methods for treating and reversing DDD in patients.

### SUMMARY OF THE INVENTION

In accordance with one or more embodiments of the present invention, the inventors investigated the potential effect of intermittent PTH (iPTH, e.g., daily injection) on LDD. It was found that iPTH effectively attenuates disc degeneration by activation of latent TGF-β in ECM during aging in mice. PTH induces NP cell expression of integrin αvβ6, which activates the TGF-β-CCN2-matrix protein signaling cascade. PTH is thus a native regulator of disc homeostasis and function during aging. These findings provide a novel mechanism of PTH signaling through NP cells to activate anabolic activity and its use for attenuating LDD and related disorders.

In accordance with an embodiment, the present invention provides a method for treatment of IVD degeneration and/or LDD in a subject having symptoms of IVD degeneration and/or LDD comprising administering to the subject an effective amount of Parathyroid hormone (PTH) or a functional fragment or analog thereof.

In accordance with an embodiment, the present invention provides a method for treatment of IVD degeneration and/or LDD in a subject having symptoms of IVD degeneration and/or LDD comprising administering to the subject an effective amount of a pharmaceutical composition comprising PTH or a functional fragment or analog thereof, and a pharmaceutically acceptable carrier.

In accordance with an embodiment, the present invention provides a method for regeneration of aged IVD in a subject comprising administering to the subject an effective amount of PTH or a functional fragment or analog thereof.

In accordance with another embodiment, the present invention provides a method for regeneration of aged IVD in a subject comprising administering to the subject an effective amount of a pharmaceutical composition comprising Parathyroid hormone (PTH) or a functional fragment or analog thereof, and a pharmaceutically acceptable carrier.

In accordance with an embodiment, the present invention provides a method for treatment of intravertebral disc (IVD) degeneration in a subject comprising administering to the subject an effective amount of Parathyroid hormone (PTH) or a functional fragment or analog thereof.

In accordance with another embodiment, the present invention provides a method for treatment of intravertebral disc (IVD) degeneration in a subject comprising administering to the subject an effective amount of a pharmaceutical composition comprising Parathyroid hormone (PTH) or a functional fragment or analog thereof, and a pharmaceutically acceptable carrier.

In accordance with an embodiment, the present invention provides a method for increasing total tissue volume of the nucleus pulposus (NP) and/or annulus fibrosus (AF) and/or cartilaginous endplates (EP) in the IVD of a subject comprising administering to the subject an effective amount of Parathyroid hormone (PTH) or a functional fragment or analog thereof.

In accordance with yet another embodiment, the present invention provides a method for reducing NP cell apoptosis in the IVD of a subject comprising administering to the subject an effective amount of Parathyroid hormone (PTH) or a functional fragment or analog thereof.

In accordance with still another embodiment, the present invention provides a method for increasing the levels of active TGF-β in the NP cells of the IVD of a subject comprising administering to the subject an effective amount of Parathyroid hormone (PTH) or a functional fragment or analog thereof.

### BRIEF DESCRIPTION OF THE DRAWINGS

FIGS. 1A-1I show IVD volume and TGF-β activity decreased during aging. (A) 3D propagation phase contrast micro-tomography (PPCT) images of IVDs of 2-month and 18-month old mice. Scale bars, 500 μm. (B) Quantitative analysis of IVD height and volume. (C) 3D upper and lower surface PPCT images showing thickness distribution among five areas of IVDs in 2-month and 18-month old mice and (D) quantitative analysis of (C). Scale bar, color code indicating degree of thickness from blue (100 μm) to red (800 μm). C: central region, R: right region, A: anterior region, L: left region, P: posterior region. (E, F): Sofranin-O staining images of IVD tissue sections showing nucleus polposus (NP) area (E) and quantitative analysis of the cell numbers in NP area and IVD histological scores of 2-month and 18-month old mice (F). Scale bars, 100 μm. (G) Immunostaining images of IVD sections showing expression of aggrecan (ACAN), CCN2 and pSmad2/3 positive cells in the NP area. Scale bars, 200 p.m. (H) Quantitative analysis of ACAN- and CCN2-positive areas as percentage of total IVD area and pSmad2/3 positive cells in NP area (Ar). Scale bars, 50 μm. (I) Western blot analysis showing pSmad2 levels in NP tissues of 2-month and 18-month old mice. All data shown as mean±s.d. *P<0.05, **P<0.01, n=8 per group. Statistical significance was determined by student t-test.

FIGS. 2A-2I show that PTH directly induces cAMP production and phosphorylation of CREB in NP cells. (A) Immunostaining images of mouse IVD sections showing PTH1R (brown) in NP cells with IgG antibody as negative control (NC). Scale bar, 100 μm. (B) Western blot analysis showing PTH1R expression in NP and AF cells with HEK293 cells as negative control (NC) and UMR-106 osteoblast-like cells as positive control (PC). (C) Lineage mapping of PTH1R expression in NP cells of notochordal origin (top yellow) using NotoCre; ROSA26-GFP mice. Scale bar, 20 μm (top) and 50 μm (bottom). NP cells stained positively for notochord origin (green) and presence of PTH1R receptor (red). Cells of notochordal origin (green) and PTH1R positive cells (red) stained abundantly with in NP area of disc tissue co-localization (yellow). (D) ELISA analysis of cellular cAMP levels in NP cells with PTH treatment. (E) Immunostaining of IVD sections showing pCREB-positive cells in NP area at different time points with PTH treatment. Scale bars, 100 μm. (F) Percentage of pCREB-positive cells versus total NP cells with PTH treatment. (G) Western blot analysis of pCREB levels in NP cells with PTH treatment. (H) Western blot analysis of PTH1R expression in human NP specimens at different ages. (I) qRTPCR of PTH1R mRNA levels in NP tissues from young and aging patients shown as fold changes. All data shown as mean±s.d. *P<0.05, **P<0.01. n=8 per group. Statistical significance was determined by student t-test.

FIGS. 3A-3S depict iPTH attenuated disc degeneration by inducing integrin αvβ6 expression in activation of TGF-β. (A) 3D PPCT images of IVDs with iPTH (1-34) injection of 40 μg/kg daily or vehicle of 18-month old mice, 5 days per week for 8 weeks. Scale bars, 500 μm. (B) Quantitative analysis of mouse IVD height and IVD volume. (C, D) 3D PPCT images and quantitative analysis showing thickness distribution of five regions of IVD with iPTH or vehicle. Color code indicating degree of thickness from blue (100 μm) to red (800 μm). (E, F) MRI scan of mouse lumbar spine showing signal intensity of the discs (yellow arrow) of 18-month old mice with iPTH or vehicle in comparison with MRI scan of 2-month old mice and quantitative measurements of the disc signal intensity (F). Scale bars, 1 mm. (G, H) Sofranin-O staining images of IVD sections showing NP area of 18-month old mice with iPTH or vehicle (G) and quantitative analysis of cell numbers in NP area and IVD histological scores (H). Scale bars, 100 μm. (I) Immunostaining images of IVD sections showing expression of ACAN, CCN2 and pSmad2/3 positive cells in NP area. Scale bars, 200 μm. (J) Quantitative analysis of the ACAN-, CCN2-positive areas and the number of pSmad2/3 positive cells as a percentage of total IVD area (Ar). (K, L) ELISA analysis of total and active TGF-β levels from NP tissue in 18-month old mice with iPTH or vehicle. (M) Western blot analysis showing pSmad2 levels on in NP tissues from 18-month old mice injected with PTH or vehicle in comparison with that of NP tissues from 2-month old. (N) Immunostaining images showing various integrin expressions in IVD tissue from 18-month old mice injected with PTH or vehicle and quantitative analysis (0). Scale bar 50 μm. (P) qRT-PCR analysis of the mRNA levels of various integrin in NP tissue from 18-month old mice injected with PTH or vehicle. Results reported as fold change. (Q) Western blot analysis of integrin β6 expression in NP cells of 18-month old mice at differenttime points post after PTH injection (PTH1-34, 100 nM). (R, S) Chromatin immunoprecipitation assay with 4 different potential pCREB binding sites (Primers 1, 2, 3 and 4) in the (36 integrin promoter. All data are reported as the mean±s.d. *P<0.05, **P<0.01. n=8 per group. Statistical significance was determined by one-way ANOVA and student t-test.

FIGS. 4A-4M show acceleration of disc degeneration in PTH1R knockout mice specifically in NP cells. (A) Immunostaining images showing no PTH1R expression in NP tissue of PTH1R deficient mice (PTH1R^{-/-}) relative their wild type littermates (PTH1R^{+/+}). IVDs (top); NP (bottom). Scale bar 50 μm. (B) 3D PPCT images of IVD thickness distribution in PTH1R^{-/-} mice at different ages compared to PTH1R^{+/+} mice. Scale bar, color code indicating the degrees of thickness from blue (100 μm) to red (800 μm). (C) Quantitative analysis of IVD volume in PTH1R^{-/-} mice at different ages compared to PTH1R^{+/+} mice. (D, E) Images of the 3D finite element analysis model for testing spine flexibility in PTH1R-deficient mice at 6- and 12-month old mice. (D) The upper surface of L3 and bottom surface of L4 were fixed with rigid bars to mimic the loading of IVD for flexibility measurement. (E) For each model, the torque loading was applied to simulate motion in four different directions; dorsiflexion, anteflexion, left and right lateral flexion measurement. (F) Quantitative analysis of spine flexibility in PTH1R deficient mice at 6- and 12-month old of age. (G) Schematic representation of the unstable spine model generated by resection of the third and fourth lumbar spinous processes along with the supraspinous and interspinous ligaments from second to fifth lumbar vertebra. (H) 3D PPCT images of IVD thickness distribution in PTH1R-deficient mice at different time points post-surgery compared with those of PTH1R^{+/+} mice. (I) Quantitative analysis of IVD volume of (H). (J) 3D PPCT images showing IVD thickness distribution in 12-month old PTH1R-deficient mice or PTH1R^{+/+} mice with iPTH or vehicle. (K) Quantitative analysis of IVD volume in (J). Western blot analysis of pSmad2/3, CCN2 and ACAN in NP tissue from PTH1R-deficient mice treated with iPTH or vehicle. (M) qRT-PCR analysis of the mRNA expression levels of CCN2 and ACAN in NP tissue from PTH1R-deficient mice or PTH1R^{+/+} mice with iPTH or vehicle. Results reported as fold change. *P<0.05, **P<0.01. n=8 per group. Statistical significance was determined by one-way ANOVA and student t-test. All data are reported as the mean±s.d.

FIGS. 5A-5N show PTH and mechanical stress stimulated transport PTH1R to primary cilium of NP cells (A) Immunostaining for acetylated α-tubulin (green) and DAPI (blue) showing the length of primary cilia of NP cells from PTH1R-deficient mice or PTH1R^{+/+} mice (B) Quantitative measurements of primary cilia length of (A). (C) Immunostaining for acetylated α-tubulin or PTH1R showing that PTH stimulated transport of PTH1R to primary cilia of NP cells. (D) Quantitative analysis of PTH1R intensity in cilia of (C). (E) Immunostaining for DEPI, pCREB or acetylated α-tubulin showing that PTH stimulated phosphorylation of CREB at primary cilia of NP cells. (F) Quantitative analysis of pCREB intensity in cilia of (E). (G, H) Coimmunoprecipitation of cell lysates from NP cells treated with PTH or vehicle using antibody against pCREB and blotted with PTH1R (G) or using antibody against PTH1R and blotted with pCREB (H) showing the interaction between PTH1R and pCREB in the acetylated α-tubulin extracts. (I) Immunostaining for acetylated α-tubulin or PTH1R showing that shear stress stimulated transport of PTH1R to primary cilia of NP cells. (J) Quantitative analysis of PTH1R intensity in cilia of (I). (K) Immunostaining of NP cells treated with pallidin siRNA or control siRNA for acetylated α-tubulin or PTH1R showing that PTH-stimulated transport of PTH1R to primary cilium of NP cells was inhibited. Scale bar=10 μm. (L) Quantitative analysis of PTH1R intensity in cilia of (K). (N) Western blot analysis of PTH-induced pCREB levels in NP cells treated with pallidin siRNA or control siRNA.*P<0.05, **P<0.01. n=8 per group. Statistical significance was determined by one-way ANOVA and student t-test. All data are reported as the mean±s.d.

FIGS. 6A-6H show Primary cilia regulates PTH signaling in NP cells for disc anabolic activity. (A) 3D PPCT and immunostaining images showing that PTH effect on IVDs diminished in IFT88^{-/-} mice. PPCT Scale bar, 500 μm. Saf-o Scale bars, 100 μm. (B) Quantitative analysis of IVD volume and (C) IVD histological scores of (A). (D) Western blot analysis of pCREB in NP cells isolated from IFT88^{-/-} or IFT88^{+/+} mice injected with iPTH or vehicle with or without shear stress. (E) Immunostaining for pSmad2/3 (brown), CCN2 and ACAN (red) with DAPI (blue) of in 2-months IFT88^{-/-} or IFT88^{+/+} mice treated with PTH or vehicle. pSmad2/3 and CCN2 Scale bar, 50 μm; ACAN Scale bar, 100 μm. (F, G and H) Quantitative analysis of percentages of pSmad2/3 positive cells in NP area and CCN2 and ACAN positive area of total IVD area. *P<0.05, **P<0.01. n=8, per group. Statistical significance was determined by one-way ANOVA and student t-test. All data are reported as the mean±s.d.

FIGS. 7A-7E depict 3D visualization of intervertebral disc by Propagation phase contrast micro-tomography (PPCT) based on the Synchroton radiation. (A) Schematic depiction of PPCT scanning at the BL13W1 biomedical beamline in the Shanghai Synchrotron Radiation Facility (SSRF) in China. The sample were fixed on a rotation stage. The inner structure was recorded by the image detector located at a relevant distance to the sample stage after transmission of monochromatic synchrotron radiation x-ray beam through the sample. (B) Coronal, transverse and sagittal views of the intervertebral disc were detected by PPCT; these views of the disc are difficult to delineate with conventional μCT. (C) The intensity distribution of the correspondence lines marked in (B). The blue line (I, II and III) refer to μCT, whereas the red line (1, 2, 3) refer to PPCT. (D) Intact 3D image of the L3-4 segment. The vertebra (VB), endplate (EP) and IVD could be visualized separately and from multiple perspectives. The 3D thickness distribution could be defined using different color coding. Scale bar=500 μm.

FIGS. 8A-8D show the dose screen of intermittent PTH effect on intervertebral disc morphology. (A) The 3D thickness distribution of IVD after iPTH treatment with 20 μg/kg/d, 40 μg/kg/d and 80 μg/kg/d of human PTH (1-34). (B) Quantitative analysis of IVD volume after iPTH treatment. (C). 3D image of L4 vertebra from 2-months-old mice and 18-months-old mice looking at the microarchitecture with or without PTH. (D). Quantitative analysis of trabecular bone volume fraction (BV/TV), trabecular thickness (Tb. Th), trabecular number (Tb. N) and trabecular separation (Tb. Sp) after iPTH treatment. *P<0.05, **P<0.01. Statistical significance was determined by one-way ANOVA and student t-test. All data are reported as the mean±s.d.

FIGS. 9A-9C show the 3D finite element analysis model for IVD flexibility testing. (A) The upper surface of L3 and bottom surface of L4 were fixed with rigid bars to mimic the loading of IVD for flexibility measurement. (B) For each model, we applied torque loading to simulate motion in four different directions; dorsiflexion, anteflexion, left and right lateral flexion measurement. (C) Sample 3D finite element model.

### DETAILED DESCRIPTION OF THE INVENTION

The IVD can be viewed as an integrated functional joint unit that can be separated macroscopically into at least three distinct components: 1) the nucleus pulposus (NP) of notochord origin, representing a centrally located gelatinous homogenous mass (in juvenile discs); 2) the annulus fibrosus (AF) populated by fibrochondrocyte-like cells of mesenchymal origin, consisting of concentrically organized layers of collagen fibrils and containing the nucleus pulposus; 3) the cartilaginous endplates (EP), which separate the NP and AF from the adjacent vertebral bone. Any disturbance of the integrity and interplay of one of the three structures can result in a compromised function of the IVD unit. It is the NP that is thought to be required for generation and maintenance of the disc's structural integrity and is the first structure to be affected during degeneration.

Originated purely from the notochord anlage, the NP is enveloped by the AF and sandwiched by the EP. NP degeneration is characterized by a cell-driven imbalance between matrix synthesis and degradation. Physiologically in juvenile discs, NP cells synthesize proteoglycans, aggrecan and biglycan with negative charged side chains to retain large quantity of water in balance the compressive stress from the EP. With increase of age, calcium pyrophosphate dihydrate crystal deposits, a visible manifestation of a metabolic abnormality, are found frequently in the AF and cartilage EP of elderly patients with degenerative disc disease. This compromises the nutrient diffusion from EP to NP through capillary buds leading to a microenvironment that becomes increasingly acidic through the buildup of lactic acid. This decreases the ability of nucleus pulposus cells to produce ECM but does not inhibit the production of degradative enzymes, such as MMPs and ADAMTS. Aggrecan degradation becomes the most significant change leading to decrease of water content in the NP, narrowing of the disc space and accelerating the sclerosis of EP. This forms a negative feedback circuit and aggravates the disc degeneration. The NP eventually loses its ability to distribute the compressive forces between the vertebral bodies, which are non-uniformly transferred to the AF, generating areas of increased pressure and risk of microtrauma. This altered force distribution and micro-trauma results in tears and fissures along the AF and ultimately loss of disc height. It is thought that the maintenance of the NP matrix in the adult human disc is dependent on the functional integrity of the cartilage EP, and the loss of ECM producing cells accounts for IVD degeneration.

In accordance with an embodiment, the present invention provides a method for treatment of IVD degeneration and/or LDD in a subject having symptoms of IVD degeneration and/or LDD comprising administering to the subject an effective amount of Parathyroid hormone (PTH) or a functional fragment or analog thereof.

In accordance with an embodiment, the present invention provides a method for treatment of IVD degeneration and/or LDD in a subject having symptoms of IVD degeneration and/or LDD comprising administering to the subject an effective amount of a pharmaceutical composition comprising PTH or a functional fragment or analog thereof, and a pharmaceutically acceptable carrier.

In accordance with an embodiment, the present invention provides a composition comprising an effective amount of Parathyroid hormone (PTH) or a functional fragment or analog thereof for use in the treatment of IVD degeneration and/or LDD in a subject.

In accordance with an embodiment, the present invention provides a composition comprising an effective amount of Parathyroid hormone (PTH) or a functional fragment or analog thereof for use in regeneration of aged intravertebral discs (IVD) in a subject.

In accordance with an embodiment, the present invention provides a composition comprising an effective amount of Parathyroid hormone (PTH) or a functional fragment or analog thereof for use in increasing total tissue volume of the nucleus pulposus (NP) and/or anulus fibrosus (AF) and/or cartilaginous endplates (EP) in the IVD of a subject.

In accordance with an embodiment, the present invention provides a composition comprising an effective amount of Parathyroid hormone (PTH) or a functional fragment or analog thereof for use in reducing NP cell apoptosis in the IVD of a subject.

In accordance with an embodiment, the present invention provides a composition comprising an effective amount of Parathyroid hormone (PTH) or a functional fragment or analog thereof for use in increasing the levels of active TGF-β in the NP cells of the IVD of a subject.

In accordance with some embodiments, the parathyroid hormone administered to a subject can be in the form of a composition or solution may incorporate the full length, 84 amino acid form of parathyroid hormone, particularly the human form, hPTH (1-84), obtained either recombinantly, by peptide synthesis or by extraction from human fluid. See, for example, U.S. Pat. No. 5,208,041, incorporated herein by reference. The amino acid sequence for hPTH (1-84) is reported by Kimura et al. in Biochem. Biophys. Res. Comm., 114(2):493.

In alternative embodiments compositions or solutions comprising PTH may also incorporate as active ingredients, functional fragments or portions or variants of fragments of human PTH or of rat, porcine or bovine PTH that have human PTH activity as determined in the ovariectomized rat model of osteoporosis reported by Kimmel et al., Endocrinology, 1993, 32(4):1577.

The parathyroid hormone functional fragments or portions desirably incorporate at least the first 28 N-terminal residues, such as PTH(1-28), PTH(1-31), PTH(1-34), PTH(1-37), PTH(1-38) and PTH(1-41). Alternatives in the form of PTH variants incorporate from 1 to 5 amino acid substitutions that improve PTH stability and half-life, such as the replacement of methionine residues at positions 8 and/or 18 with leucine or other hydrophobic amino acid that improves PTH stability against oxidation and the replacement of amino acids in the 25-27 region with trypsin-insensitive amino acids such as histidine or other amino acid that improves PTH stability against protease. Other suitable forms of PTH include PTHrP, PTHrP(1-34), PTHrP(1-36) and analogs of PTH or PTHrP that activate the PTH1 receptor. These forms of PTH are embraced by the term “parathyroid hormone” as used generically herein. The hormones may be obtained by known recombinant or synthetic methods, such as described in U.S. Pat. Nos. 4,086,196 and 5,556,940, incorporated herein by reference.

In one embodiment, the methods of the present invention the PTH is human PTH(1-34), also known as teriparatide. Stabilized solutions of human PTH(1-34), such as recombinant human PTH(1-34) (rhPTH(1-34)), that can be employed in the present methods, including crystalline forms, are described in U.S. Pat. No. 6,590,081, incorporated herein by reference.

The term, “amino acid” includes the residues of the natural α-amino acids (e.g., Ala, Arg, Asn, Asp, Cys, Glu, Gln, Gly, His, Lys, Ile, Leu, Met, Phe, Pro, Ser, Thr, Trp, Tyr, and Val) in D or L form, as well as β-amino acids, synthetic and non-natural amino acids. Many types of amino acid residues are useful in the polypeptides and the invention is not limited to natural, genetically-encoded amino acids. Examples of amino acids that can be utilized in the peptides described herein can be found, for example, in Fasman, 1989, CRC Practical Handbook of Biochemistry and Molecular Biology, CRC Press, Inc., and the reference cited therein. Another source of a wide array of amino acid residues is provided by the website of RSP Amino Acids LLC.

Reference herein to “derivatives” includes parts, fragments and portions of the PTH peptides. A derivative also includes a single or multiple amino acid substitution, deletion and/or addition. Homologues include functionally, structurally or stereochemically similar peptides from the naturally occurring peptide or protein. All such homologs are contemplated by the present invention.

Analogs and mimetics include molecules which include molecules which contain non-naturally occurring amino acids or which do not contain amino acids but nevertheless behave functionally the same as the PTH peptide. Natural product screening is one useful strategy for identifying analogs and mimetics.

Examples of incorporating non-natural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, omithine, sarcosine, 4-amino-3-hydroxy-6-methylheptanoic acid, 2-thienyl alanine and/or D-isomers of amino acids. A partial list of known non-natural amino acid contemplated herein is shown in Table 1.

TABLE 1
Non-natural Amino Acids

| Non-conventional | |
|---|---|
| amino acid | Code |
| α-aminobutyric acid | Abu |
| α-amino-a-methylbutyrate | Mgabu |
| aminocyclopropane- | Cpro |
| carboxylate | |
| aminoisobutyric acid | Aib |
| aminonorbomyl- | Norb |
| carboxylate | |
| cyclohexylalanine | |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl) | Nnbhm |
| carbamylmethyl)glycine | |
| 1-carboxy-1-(2,2-diphenyl- | Nmbc |
| ethylamino)cyclopropane | |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| Chexa L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-γ-aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl)glycine | |

Analogs of the subject peptides contemplated herein include modifications to side chains, incorporation of non-natural amino acids and/or their derivatives during peptide synthesis and the use of crosslinkers and other methods which impose conformational constraints on the peptide molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of Ca and Na-methylamino acids, introduction of double bonds between C_{α} and C_{β} atoms of amino acids and the formation of cyclic peptides or analogues by introducing covalent bonds such as forming an amide bond between the N and C termini, between two side chains or between a side chain and the N or C terminus.

The present invention further contemplates small chemical analogs of the naturally occurring Pep moiety. Chemical analogs may not necessarily be derived from the peptides themselves but may share certain conformational similarities. Alternatively, chemical analogs may be specifically designed to mimic certain physiochemical properties of the peptides. Chemical analogs may be chemically synthesized or may be detected following, for example, natural product screening.

Parathyroid hormone is an FDA-approved anabolic therapy for osteoporosis, and is capable of acting directly on bone matrix secreting osteoblasts and well known for its function in regulation of skeletal homeostasis. The parathyroid gland, the main production site of the PTH, evolved in amphibians and represents the transition of aquatic to terrestrial life, adapting terrestrial locomotion from aquatic vertebrates. PTH has been shown to activate resting cells for the skeletal integrity and remodeling such as converting lining cells to active osteoblasts and orchestrates signaling of local factors, including (but not limited to) TGFβ, Wnts, BMP and IGF-1. Thus, PTH regulates cellular activities-including those of MSCs, T cells, and other PTH-responsive cells-in the microenvironment to integrate systemic control of tissue homeostasis. Moreover, small blood vessels were spatially relocated closer to sites of new bone formation in PTH-stimulated bone remodeling. The closer proximity of blood vessels allows efficient delivery of nutrients for the skeletal tissue homeostasis, particularly for non-vasculature IVD.

In accordance with an embodiment, the present invention provides a method for regeneration of aged intravertebral discs (IVD) in a subject comprising administering to the subject an effective amount of PTH or a functional fragment or analog thereof.

In reference to the parent PTH polypeptide, the functional portion can comprise, for instance, about 90%, 95%, or more, of the PTH polypeptide.

The functional portion of the PTH can comprise additional amino acids at the amino or carboxy terminus of the portion, or at both termini, which additional amino acids are not found in the amino acid sequence of the PTH polypeptide. Desirably, the additional amino acids do not interfere with the biological function of the functional portion.

Included in the scope of the invention are functional variants of the inventive polypeptides, and proteins described herein. The term “functional variant” as used herein refers to PTH polypeptide, or protein having substantial or significant sequence identity or similarity to PTH polypeptide, or protein, which functional variant retains the biological activity of PTH polypeptide, or protein of which it is a variant. In reference to the parent PTH polypeptide, or protein, the functional variant can, for instance, be at least about 30%, 50%, 75%, 80%, 90%, 98% or more identical in amino acid sequence to the PTH polypeptide, or protein.

The functional variant can, for example, comprise the amino acid sequence of the PTH polypeptide, or protein with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Functional variants can also include extensions of the PTH polypeptide. For example, a functional variant of the PTH polypeptide can include 1, 2, 3, 4 and 5 additional amino acids from either the N-terminal or C-terminal end of the PTH polypeptide.

Alternatively or additionally, the functional variants can comprise the amino acid sequence of the PTH polypeptide, or protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the PTH polypeptide, or protein.

The PTH polypeptide, or protein can consist essentially of the specified amino acid sequence or sequences described herein, such that other components of the functional variant, e.g., other amino acids, do not materially change the biological activity of the functional variant.

The PTH polypeptide used in the methods of the invention (including functional portions and functional variants) of the invention can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

PTH polypeptides and proteins used in the methods of the invention (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

When PTH polypeptides and proteins used in the invention (including functional portions and functional variants) are in the form of a salt, preferably, the polypeptides are in the form of a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

PTH polypeptides and/or proteins used in the methods of the invention (including functional portions and functional variants thereof) can be obtained by methods known in the art. Suitable methods of de novo synthesizing polypeptides and proteins are described in references, such as Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwoood et al., Oxford University Press, Oxford, United Kingdom, 2000; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, N Y, 1994. Further, some of the PTH polypeptides and proteins of the invention (including functional portions and functional variants thereof) can be isolated and/or purified from a source, such as a plant, a bacterium, an insect, a mammal, e.g., a rat, a human, etc. Methods of isolation and purification are well-known in the art. Alternatively, PTH polypeptides, and/or proteins described herein (including functional portions and functional variants thereof) can be commercially synthesized by companies, such as Synpep (Dublin, Calif.), Peptide Technologies Corp. (Gaithersburg, Md.), and Multiple Peptide Systems (San Diego, Calif.). In this respect, the PTH polypeptides, and proteins can be synthetic, recombinant, isolated, and/or purified.

Included in the scope of the invention are conjugates, e.g., bioconjugates, comprising any of the PTH polypeptides or proteins (including any of the functional portions or variants thereof), nucleic acids, recombinant expression vectors, host cells, populations of host cells, or antibodies, or antigen binding portions thereof. Conjugates, as well as methods of synthesizing conjugates in general, are known in the art (See, for instance, Hudecz, F., Methods Mol. Biol. 298: 209-223 (2005) and Kirin et al., Inorg Chem. 44(15): 5405-5415 (2005)).

In accordance with an embodiment, the present invention provides a method for treatment of IVD degeneration in a subject comprising administering to the subject an effective amount of PTH or a functional fragment or analog thereof.

As used herein, the term “IVD degeneration” or “degenerative disk disease” can mean the narrowing of IVD space, or decreasing extracellular matrix and cell numbers in either or both NP and AF, and also can mean the metabolic failure of matrix production in the NP and EP. In some embodiments, degeneration can include decreasing numbers of pSmad2/3+ cells in the NP and AF.

As used herein, the term “treat,” as well as words stemming therefrom, includes diagnostic and preventative as well as disorder remitative treatment. Furthermore, the treatment or prevention provided by the inventive method can include treatment or prevention of one or more conditions or symptoms of the disease, e.g., diarrhea, being treated or prevented. Also, for purposes herein, “prevention” can encompass delaying the onset of the disease, or a symptom or condition thereof.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

In accordance with an embodiment, the present invention provides a method for increasing total tissue volume of the nucleus pulposus (NP) and/or anulus fibrosus (AF) and/or cartilaginous endplates (EP) in the IVD of a subject comprising administering to the subject an effective amount of PTH or a functional fragment or analog thereof.

In accordance with yet another embodiment, the present invention provides a method for reducing NP cell apoptosis in the IVD of a subject comprising administering to the subject an effective amount of PTH or a functional fragment or analog thereof.

In accordance with still another embodiment, the present invention provides a method for increasing the levels of active TGF-β in the NP cells of the IVD of a subject comprising administering to the subject an effective amount of PTH or a functional fragment or analog thereof.

An effective amount of PTH or a functional fragment or analog thereof, to be employed therapeutically will depend, for example, upon the therapeutic and treatment objectives, the route of administration, the age, condition, and body mass of the subject undergoing treatment or therapy, and auxiliary or adjuvant therapies being provided to the subject. Accordingly, it will be necessary and routine for the practitioner to titer the dosage and modify the route of administration, as required, to obtain the optimal therapeutic effect. A typical daily dosage of PTH or a functional fragment or analog thereof might range from about 0.1 mg/kg to up to about 100 mg/kg or more, preferably from about 0.1 to about 10 mg/kg/day depending on the above-mentioned factors. Typically, the clinician will administer PTH or a functional fragment or analog thereof until a dosage is reached that achieves the desired effect. The progress of this therapy is easily monitored by conventional assays.

The dosage ranges for the administration of PTH peptides or derivatives thereof, are those large enough to produce the desired effect in which the symptoms of the malignant disease are ameliorated. The dosage should not be so large as to cause adverse side effects, such as unwanted cross-reactions, anaphylactic reactions, and the like. Generally, the dosage will vary with the age, condition, sex and extent of disease of the patient and can be determined by one of skill in the art. The dosage can be adjusted by the individual physician in the event of any complication.

As a non-limiting example, treatment of subjects can be provided as a one-time or periodic dosage of PTH peptides or derivatives thereof at about 0.1 mg to 100 mg/kg such as 0.1 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, or repeated doses.

Specifically, the PTH peptides or derivatives thereof may be administered at least once a day over the course of several weeks. In one embodiment, the pharmaceutical compositions are administered at least once a day over several weeks to several months. In another embodiment, the pharmaceutical compositions are administered several times a week over four to eight weeks, such as, for example, 2, 3, 4, 5 times a week. In yet another embodiment, the pharmaceutical compositions are administered once a week over four weeks or longer.

In accordance with another embodiment, the present invention provides a method for regeneration of aged IVD in a subject comprising administering to the subject an effective amount of a pharmaceutical composition comprising Parathyroid hormone (PTH) or a functional fragment or analog thereof, and a pharmaceutically acceptable carrier.

In accordance with an embodiment, the present invention provides a pharmaceutical composition comprising the peptides heretofore described above, and a pharmaceutically acceptable carrier.

In accordance with another embodiment, the present invention provides a pharmaceutical composition comprising the peptides heretofore described above, and a second therapeutic agent, and a pharmaceutically acceptable carrier.

With respect to peptide compositions described herein, the pharmaceutically acceptable carrier can be any of those conventionally used, and is limited only by physico-chemical considerations, such as solubility and lack of reactivity with the active compound(s), and by the route of administration. The carriers described herein, for example, vehicles, adjuvants, excipients, and diluents, are well-known to those skilled in the art and are readily available to the public. It is preferred that the carrier be one which is chemically inert to the active agent(s), and one which has little or no detrimental side effects or toxicity under the conditions of use. Examples of the carriers include soluble carriers such as known buffers which can be physiologically acceptable (e.g., phosphate buffer) as well as solid compositions such as solid-state carriers or latex beads.

The carriers or diluents used herein may be solid carriers or diluents for solid formulations, liquid carriers or diluents for liquid formulations, or mixtures thereof.

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

For liquid formulations, pharmaceutically acceptable carriers may be, for example, aqueous or non-aqueous solutions, or suspensions. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Formulations suitable for parenteral administration include, for example, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

It will be appreciated by one of skill in the art that, in addition to the above-described pharmaceutical compositions, the invention can be formulated as inclusion complexes, such as cyclodextrin inclusion complexes, or liposomes.

In addition, in an embodiment, the compositions comprising PTH or derivatives thereof, may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmelose sodium, crospovidone, guar gum, sodium starch glycolate), buffers (e.g., Tris-HCl., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., cremophor, glycerol, polyethylene glycerol, benzlkonium chloride, benzyl benzoate, cyclodextrins, sorbitan esters, stearic acids), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweetners (e.g., aspartame, citric acid), preservatives (e.g., thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates), and/or adjuvants.

The choice of carrier will be determined, in part, by the particular peptide containing compositions, as well as by the particular method used to administer the composition. Accordingly, there are a variety of suitable formulations of the pharmaceutical compositions of the invention. The following formulations for parenteral, subcutaneous, intramuscular, and interperitoneal administration are exemplary, and are in no way limiting. More than one route can be used to administer the compositions of the present invention, and in certain instances, a particular route can provide a more immediate and more effective response than another route.

Injectable formulations are in accordance with the invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Trissel, 15th ed., pages 622-630 (2009)).

As used herein the term “pharmaceutically active compound” or “therapeutically active compound” means a compound useful for the treatment or modulation of a disease or condition in a subject suffering therefrom. Examples of pharmaceutically active compounds can include any drugs known in the art for treatment of disease indications.

Generally, when the PTH polypeptides or derivatives thereof, are administered together with additional therapeutic agents, lower dosages can be used. PTH or derivatives thereof, can be administered parenterally by injection or by gradual perfusion over time. PTH polypeptides or derivatives thereof, can be administered intraperitoneally, intramuscularly, subcutaneously, intracavity, or transdermally, alone or in combination with effector cells. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Preservatives and other additives may also be present such as, for example, antimicrobials, anti-oxidants, chelating agents, and inert gases and the like.

### EXAMPLES

Human Subjects.

After approval by the Institutional Review Board of Johns Hopkins University, lumbar disc specimens were collected from five patients with different ages who were undergoing discectomy for lumbar intervertebral disc degeneration. The specimens were processed for Western blot examination.

Mice.

For ageing induced IVD degeneration model, 2 and 16 months-old C57BL/6J WT male mice were purchased from Charles River. We purchased (ROSA) 26Sortm1Sor/J mice from the Jackson Laboratory. Mice with floxed PTH1R (PTH1Rflox/flox) were obtained from the lab of Dr. Henry Kronenberg. We obtained the NotoCre mice strain from Dr. Cheryle A. Seguin.

Mice carrying the PTH1R gene flanked by loxP sites (PTH1R^{flox/flox}) were mated with Noto^{Cre} mice to generate mice bearing Noto^{Cre} and a floxed PTH1R allele in their germline. These mice were backcrossed to homozygous floxed mice (Noto^{Cre/+}/PTH1R^{flox/+}::PTH1R^{flox/flox}) to generate mice with inactivation of both alleles in notochord derived cells (genotype Noto^{+/+}::PTH1R^{flox/flox}). Homozygous disruption of the Noto locus is perinatally lethal; viable offspring have genotypes of either Noto^{Cre/+}:: PTH1R^{flox/flox} (mice with PTH1R conditional deletion in Noto lineage cells are referred to as “PTH1R^{-/+}” in the text) or Noto^{+/+}:: PTH1R^{flox/flox} (wild-type littermates hereinafter referred to as “PTH1R^{+/+}” in the text). The genotype of transgenic mice was determined by PCR analyses of genomic DNA isolated from mouse tails. The floxed PTH1R allele was identified with primers lox1F (5-TGGACGCAGACGATGTCTTTACCA-3) (SEQ ID NO: 1) and lox1R (5-ACATGGCCATGCCTGGGTCTGAGA-3) (SEQ ID NO: 2). The genotyping for the Cre transgene was performed by PCR with the primers Cre F (5-CAAATAGCCCTGGCAGAT-3) (SEQ ID NO: 3) and Cre R (5-TGATACAAGGGACATCTTCC-3) (SEQ ID NO: 4). We generated Noto^{Cre/+}:: ROSA26-lacZ^{flox/flox} by crossing Noto mice with mice homozygous with a loxP-flanked DNA stop sequence, preventing expression of the downstream lacZ gene. IFT88^{flox/flox} mouse model has been generated as descripted. A NP tissue-specific primary cilia knock-out (KO) mouse line was generated by crossing NotoCre mice with IFT88^{flox/flox} mice, in which the primary cilia were deleted from the NP tissue. The loxP IFT88 allele was identified with the primers lox2F (5′-GACCACCTTTTTAGCCTCCTG-3′) (SEQ ID NO: 5) and lox2R (5′-AGGGAAGGGACTTAGGAATGA-3′) (SEQ ID NO: 6).

For Lumbar Spine instability mouse model (LSI): 2 month-old PTH1R^{+/+} and PTH1R^{-/-} male mice were used for this experiment. After anesthetizing with ketamine and xylazine, mice were operated by resection of the Lumbar 3rd -Lumbar 4th (L3-L4) spinous processes along with the supraspinous and interspinous ligaments to induce instability of lumbar spine. Mice were euthanized at 0, 2, 4, and 8 weeks after the surgery (n=8 per group).

For the dosage screening experiments, 17 month old male C57BL/6J WT mice were assigned into four groups treated with different doses of PTH or vehicle; 20 μg/kg/d, 40 μg/kg/d and 80 μg/kg/d of human PTH (1-34) (Bachem Calif., Inc. King of Prussia, Pa., USA.) or vehicle groups. We chose the optimal dosage of PTH (1-34) for the rest of the experiment.

The 11 month-old PTH1R^{+/+} and PTH1R^{-/-} male mice were randomized into four groups: PTH1R^{+/+}+PTH (1-34), PTH1R^{+/+}+Vehicle, PTH1R^{-/-}+PTH (1-34) and PTH1R^{-/-}+Vehicle groups (n=8 per group). The 11 month-olds IFT88^{+/+} and IFT88^{-/-} male mice were randomized into four groups: IFT88^{+/+}+PTH (1-34), IFT88^{+/+}+Vehicle, IFT88^{-/-}+PTH (1-34) and IFT88^{-/-}+Vehicle groups (n=8 per group). Mice were subcutaneously injected with either PTH (1-34) or vehicle (1 mM acetic acid in phosphate buffered saline (PBS) with equivalent volume of PTH) daily, 5 days per week, and all mice were sacrificed 4 weeks after treatment with PTH (1-34)/vehicle.

All animals were maintained in the Animal Facility of the Johns Hopkins University School of Medicine. The experimental protocols for both species were reviewed and approved by the Institutional Animal Care and Use Committee of The Johns Hopkins University, Baltimore, Md., USA.

Primary NP Cell Isolation and Culture.

Green fluorescent protein (GFP) labeled NP cells of notochordal origin were isolated from 15-day old Noto^{Cre/+}::ROSA26-lacZ^{flox/flox} male mice as previously described. The NP cells from 15-day old PTH1R^{+/+} and PTH1R^{-/-}, IFT88^{+/+} and IFT88^{-/-} male mice. Briefly, the cells were isolated from the NP region of IVDs in the spinal column from mid thoracic to lower lumbar region and digested initially with TrypLE Express (Gibco) for 30 minutes on shaker, followed by 0.25 mg/ml Collagense-P (Roche) for another 30 hours at 37° C. The digested cells were washed twice with PBS, and cultured in α-MEM (Gibco) supplemented with 10% fetal calf serum (Atlanta Biologicals), and 1% penicillin—streptomycin (Mediatech) to 80-90% confluence at 37° C., 5% CO₂ and 5% O₂.

Primary Cilia Visualization and Co-Localization Analysis.

The localization of PTH signaling components within NP cells was investigated through immunocytochemistry after 48 hours of serum starvation (DMEM, 0.5% FBS, 1% P/S). Cells were treated with PTH (recombinant human PTH, 100 nmol, Bachem Calif., Inc. King of Prussia, Pa., USA.) or shear stress (1 dyn/cm²) with times of exposure for 30 minutes, fixed and stained for acetylated α-tubulin of primary cilia, PTH1R (Abcam, 1:100) and pCREP (Abcam, 1:500). Coverslips were mounted with Fluoroshield-DAPI. All cells were imaged using a Zeiss LSM710 META Confocal Laser Scanning microscope fitted with a 63× objective. Region of interest were selected manually using ImageJ software. The intensity profiles along the cilia have been performed by tracing a line across the length of the primary cilia and measuring intensity along this line using ImageJ software. Average intensities in the ciliary region, were measured on at least 30 ciliated cells per condition.

Western Blot Analysis and Co-Immunoprecipitation.

Western blot analyses were conducted on the protein lysates from in vitro cultured NP cells or NP tissues from mice at specific time points after PTH (1-34) treatment and human at different ages with lumbar disc degeneration. The protein extract was centrifuged, the concentration of supernatant evaluated by DC protein assay (Bio-Rad Laboratories), and then separated by SDS-PAGE and blotted on a polyvinylidene fluoride membrane (Bio-Rad Laboratories). Following incubation in specific antibodies, we detected proteins using an enhanced chemiluminescence kit (Amersham Biosciences). We used specific antibodies recognizing rabbit CTGF (CCN2) (Abcam, 1:1000), Aggrecan (ACAN) (Abcan, 1:1000), p-Smad2 (Cell Signaling Technology Inc., 1:1000), Smad2 (Cell Signaling Technology Inc., 1:1000), GAPDH (Cell Signaling Technology Inc., 1:1000), CREB (Abcam, 1:2500), pCREB (Abcam, 1:2500), PTH1R PRB-635P (Covance, 1:100), PTH1R PRB-640P (Covance, 1:500) and goat integrin β6 (Santa Cruz, 1:500) to examine the protein concentrations in the lysates. Co-immunoprecipitation analyses were conducted on the nuclear and cytoplasmic protein extraction using NE-PER Nuclear and Cytoplasmic Extraction Kit (Pierce Thermo Scientific, 78833) from in vitro cultured NP cells treated with PTH (1-34) or vehicle. PTH1R and pCREB expression were revealed with an anti-pCREB (Abcam, 1:500) and anti-PTH1R (Abcam, 1:500) antibodies.

Chromatin Immunoprecipitation (ChIP) Assay.

The ChIP assays were carried out using the Thermo Fisher ChIP Kit (catalog number: 26156). The crude homogenate from the NP cells was crosslinked with 1% formaldehyde at room temperature for 10 min. The reaction was stopped by adding glycine (0.25 M). After centrifugation, the pellet was collected and lysed in SDS lysis buffer containing protease inhibitor cocktail. The lysis was sonicated until the DNA was broken into fragments with a mean length of 200-1,000 bp. The samples were first pre-cleaned with protein G agarose and then subjected to immunoprecipitation overnight with 2 mg of rabbit antibodies against pCREB (CST, 1:50) With 2 mg of normal rabbit serum overnight at 4° C. The 10-20% of the sample for immunoprecipitation was used as an input (a positive control). After purification, the DNA fragments were amplified using Real-time PCR with the primers for (36 promoter listed in Table 1.

TABLE 1
Integrin β6 promoter sequence

| Target | | |
|---|---|---|
| gene | Forward primer (5′-3′) | Reverse primer (5′-3′) |
| Primer 1 | TGTGCTGTTCCAACCTCT (SEQ ID NO: | TTCCTGAAGAACACCCTG (SEQ ID |
| | 23) | NO: 24) |
| Primer 2 | TTGAAACGAACCCTGAAA (SEQ ID | TTCCCTAGCCTTCCTTCT (SEQ ID NO: |
| | NO: 25) | 26) |
| Primer 3 | ATTTTGGTGTAAGTTCTATG (SEQ ID | GACTATATTTCTATTGCTGTTGTGA |
| | NO: 27) | (SEQ ID NO: 28) |
| Primer 4 | TCAGCGTTACAAGACCAA (SEQ ID | TCCAGGTAGCCTCTGTTT (SEQ ID NO: |
| | NO: 29) | 30) |
| GADPH | AATGTGTCCGTCGTGGATCTGA (SEQ | AGTGTAGCCCAAGATGCCCTTC (SEQ |
| | ID NO: 31) | ID NO: 32) |

cAMP Assays and ELISA Analysis.

For cAMP assays, confluent cells were grown in 35-mm six-well plates starved overnight by incubation in serum-free α-MEM at 37° C. The cells were then treated with 100 nM human of human PTH (1-34; Bachem Calif., Inc.) for 1 h. Cellular cAMP was extracted and the concentration measured with the Biotrak enzyme immunoassay system (GE Healthare, Inc., Princeton, HJ). We determined the concentration of total and active TGF-β in the NP tissue using the ELISA Development Kit (R&D Systems) according to the manufacturer's instructions.

Quantitative RT-PCR.

After protein extraction, specimen from the same group of mice were prepared for RNA extraction using TRIzol reagent (Invitogen) according to the manufacturer's instruction. The purity of RNA was tested by measuring the absorbance at 260 and 280 nm. For qRT-PCR, two micrograms of RNA was reverse transcribed into cDNA using the SuperScript firststrand synthesis system (Invitrogen) and analyzed with SYBR Green-Master Mix (Qiagen) in the thermal cycler with two sets of primers specific for each target gene. Relative expression was calculated for each gene by the 2^{-ΔΔCT} method, with GAPDH for normalization. Primers used for qRT-PCR are listed in Table 2.

TABLE 2

| Target | | |
|---|---|---|
| gene | Forward primer (5′-3′) | Reverse primer (5′-3′) |
| ACAN | CAGATGGCACCCTCCGATAC (SEQ ID | GACACACCTCGGAAGCAGAA (SEQ ID |
| | NO: 7) | NO: 8) |
| CCN2 | CCGCCAACCGCAAGATC (SEQ ID NO: | ACCGACCCACCGAAGACA (SEQ ID |
| | 9) | NO: 10) |
| αV | ACTGTGAAGGCGCAGAATCA (SEQ ID | TGCCTCTATCCAGTCGACCA (SEQ ID |
| | NO: 11) | NO: 12) |
| β3 | CTCCCTTCTTCCCTCCCCTC (SEQ ID | ATCTCGATTACGGGACACGC (SEQ ID |
| | NO: 13) | NO: 14) |
| β5 | GCCAAGTTCCAAAGTGAGCG (SEQ ID | CTACCAGGTCCCTTAGGGCT (SEQ ID |
| | NO: 15) | NO: 16) |
| β6 | CTCACGGGTACAGTAACGCA (SEQ ID | CCACAAAAGAGCCAAAGCCC (SEQ ID |
| | NO: 17) | NO: 18) |
| β8 | TTATGCAGCAGAGCCCCATC (SEQ ID | CAAGACGGAAGTCACGGGAA (SEQ ID |
| | NO: 19) | NO: 20) |
| GADPH | AATGTGTCCGTCGTGGATCTGA (SEQ | AGTGTAGCCCAAGATGCCCTTC (SEQ |
| | ID NO: 21) | ID NO: 22) |

Immunocytochemistry, Immunofluorescence and Histomorphometry.

For immunocytochemical staining, we incubated GFP labeled NP cells co-stained with primary antibody to rabbit PTH1R PRB-635P (Covance, 1:100) for 1 h and subsequently stained with secondary antibodies conjugated with fluorescence at room temperature. At the time of euthanasia, we dissected and fixed the lumbar vertebral spine in 10% buffered formalin for 48 h, decalcified them in 10% EDTA (pH 7.4) for 14-21 days and embedded them in paraffin or optimal cutting temperature (OCT) compound (Sakura Finetek). Fourmicrometer-thick coronal-oriented sections of the L1-L6 spine were processed for safranin O and fast green staining. Sections for immunostaining was performed using a standard protocol and incubated with primary antibodies to rabbit ACAN (Abcam, 1:100), CCN2 (Abcam, 1:100), integrin β8 (Abcam, 1:200), pCREB (Abcam, 1:100) and PTH1R PRB-635P (Covance, 1:100), mouse pSmad2/3 (Santa Cruz, 1:100), integrin αvβ6 (Millipore, 1:100), integrin αvβ3 (Bioss, 1:100,), integrin αvβ5 (Bioss, 1:100) at 4° C. overnight. For immunohistochemical staining, a horse radish peroxidase-streptavidin detection system (Dako) was subsequently used to detect the immunoactivity, followed by counterstaining with hematoxylin (Sigma-Aldrich). For immunofluorescent assay, the slides were incubated with secondary antibodies conjugated with fluorescence at room temperature for 1 h while avoiding light. We used isotype-matched controls, such as polyclonal rabbit IgG (R&D Systems, AB-105-C) under the same concentrations and conditions as negative controls. We micro photographed sections to perform histomorphometric measurements on the entire area of the L3-L4 of the spine (Olympus DP71).

Quantitative histomorphometric analysis was conducted in a blinded fashion with Image-Pro Plus Software version 6.0 (Media Cybernetics Inc). IVD histological score were obtained as previously described67. Five randomly selected sections per mice in each group at L3-L4 level were chosen for quantitative histomorphometric analysis. The percentage of pSmad2/3 and pCREB positive cells was obtained by counting the number of positive staining cells to the number of total cells in the NP region. The percentage area of CCN2 and ACAN positive staining was calculated by measuring the positive area to the whole area of the L3-L4 in each group.

Propagation Phase Contrast Micro-Tomography Scanning.

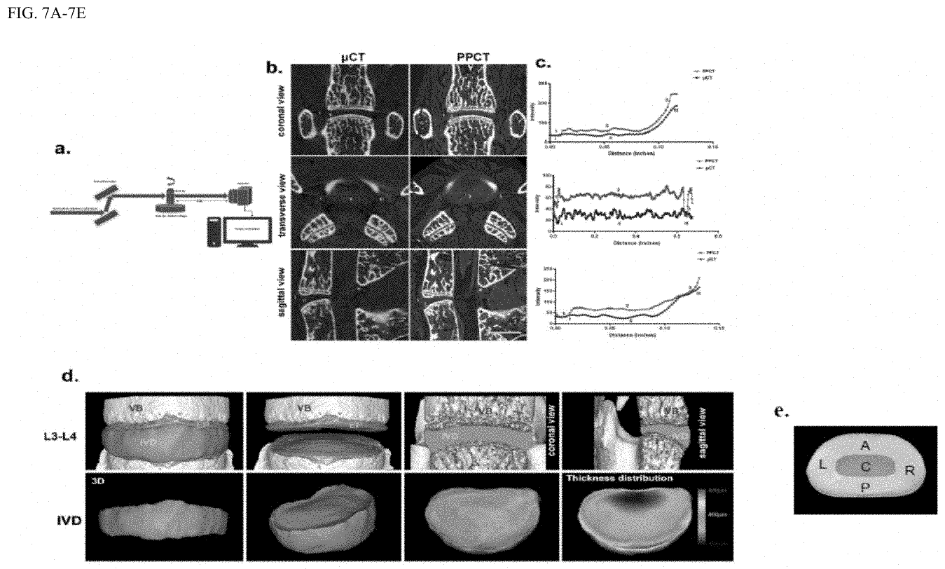

Propagation phase contrast micro-tomography (PPCT) based on the Synchrotron radiation scanning was performed at the BL13W1 biomedical beamline in the Shanghai Synchrotron Radiation Facility (SSRF) in China (FIG. 7A). To obtain high X-ray attenuation contrast images, the monochromatic X-ray energy was adjusted to 15 keV, the scanner was set at a voltage of 15 keV, exposure time set to 2.5 s, and the sample-to-detector distance (SDD) adjusted to 30 cm and a resolution of 3.7 μm per pixel. The images were reconstructed and analyzed using the GPU-CT-Reconstruction software (applied by the BL13W1 experimental station) and the VG Studio Max 3D software (version 2.1, Volume Graphics GmbH, Germany) respectively. The region of interest was defined to cover the whole L3-L4 compartment. The 3D quantitative analysis of IVDs was performed with the commercially available Image Pro Analyzer 3D software (Version 7.0, Media Cybernetics, Inc., USA). The 3D structural parameters analyzed for the mean height and volume and thickness distribution of IVDs. The trabecular bone of L4 was segmented from the bone marrow and analyzed to determine the trabecular bone volume fraction (BV/TV), trabecular thickness (Tb. Th), trabecular number (Tb. N) and trabecular separation (Tb. Sp).

PPCT Based 3D Finite Element Test.

For each model, PPCT images of one motion segment of L3-L4 were first subjected to noise elimination, and binarization was performed using thresholds obtained by discrimination analysis. Then 3D geometry models of bone tissue (including the cartilaginous endplate) and IVD were reconstructed from the segmentation results, such as shown in FIGS. 9A, C. For bone tissue model, because of the large amount of trabecular microstructure, using the geometry solid model may result in some topology errors and undesired geometric features, hence the need to preprocess with some geometry repair and optimization operations. Subsequently, the finite element mesh models can be directly discretized by 10-node quadratic tetrahedral elements from the geometry solid models, by an adaptive meshing tool in the VG Studio Max 3D software. The material properties of tissues were set according to previous studies. Finally, finite element analysis (FEA) was carried out in ABAQUS (Dassault Systèmes Americas Corp, Waltham, Mass., USA). For each model, the upper surface of L3 and bottom surface of L4 were fixed with rigid bars, such as the orange part shown in FIG. 9B, which can be precisely applied with torque loading to simulate motion in four different directions; dorsiflexion, anteflexion, left and right lateral flexion measurement. Finally, the resulting range of motion (ROM) of each motion segment was calculated.

In Vivo Micro-MRI.

In vivo Spinal IVD imaging was conducted with a horizontal 30-cm-bore 9.4T Bruker Biospec preclinical scanner equipped with custom-built, single-turn volume coil positioned orthogonal to the BO magnetic field. Anesthetization of mice was initiated with 4% isoflurane and maintained with a 2% isoflurane and oxygen mixture. Mice were placed supine on a tray and taped to minimize the motion artifacts. We acquired T2-weighted images using a relaxation enhancement (RARE) sequence with the following parameter: an echo time/repetition time (TE/TR) of 15.17 ms/3,000 ms, 35 slices at thickness of 0.35 mm, field of view (FOV) of 1.75 cm×1.75 cm and matrix size of 256×128. All T2-weighted images were processed to a final matrix size of 256×256 with an isotropic resolution of 0.068 mm pixel-1. For quantification of signal intensity as an indicator of disc tissue hydration, the region of interest at L3-L4 level in each group was selected and measured using Image-Pro Plus Software version 6.

Statistics.

Data are presented as the mean±s.d. We used unpaired, two-tailed Student's t-tests for comparisons between two groups and one-way analysis of variance (ANOVA) with Bonferroni post hoc test for multiple comparisons. All data demonstrated a normal distribution and similar variation between groups. The level of significance was set at P<0.05. All data analyses were performed using SPSS 22.0 analysis software (SPSS Inc.)

### Example 1

TGF-α activity is decreased in IVD degeneration during aging.

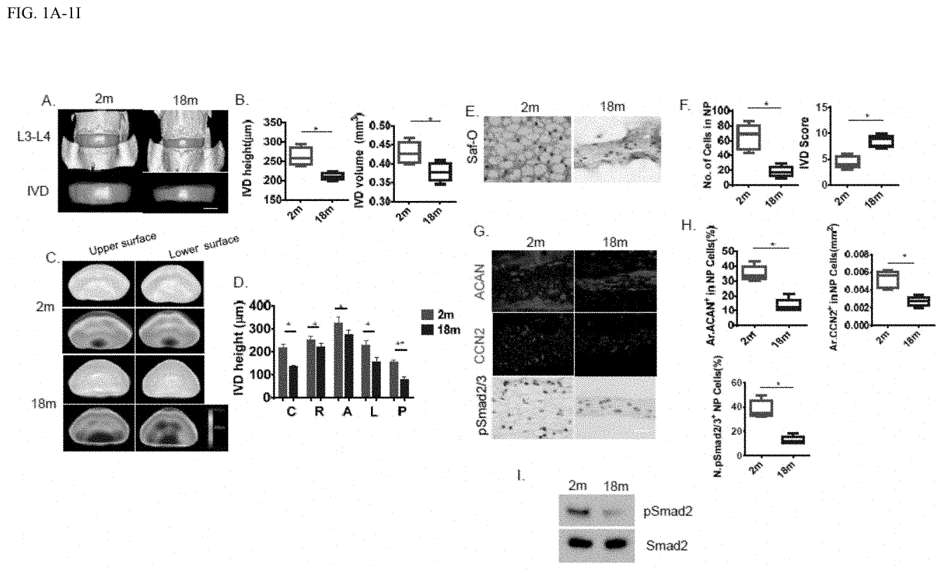

We systematically examined changes of IVD during aging. Three dimensional (3D) changes of the IVD were visualized using propagation phase contrast micro-tomography (PPCT) based on synchrotron radiation showing 3D images of the IVD with adjoining vertebra (top) and intact IVD (bottom) in 2-month and 18-month-old mice (FIG. 1A and FIG. 7). The height and volume of the IVD were significantly decreased in 18-month-old mice relative to 2-month-old mice (FIG. 1B). Similarly, thickness distribution within five different IVD areas in 3D decreased with aging (FIGS. 1C, D and FIG. 7), especially the posterior area. The number of NP cells also significantly decreased as visualized by Safranin-O staining of IVD sections in 18-month-old mice with increase of IVD score as indication of degeneration (FIGS. 1E, F). Immunohistochemical staining demonstrated that levels of aggrecan (ACAN), connective tissue growth factor (CTGF/CCN2)⁺, and pSmad2/3⁺ cells in the IVD significantly decreased in 18-month-old mice relative to 2 month-old mice (FIGS. 1G, H). Western blot analysis validated the decrease of pSmad2/3 in the 18-month-old mice (FIG. 1I). Our results show that decrease of active TGF-β is associated with its downstream anabolic CCN2-ECM cascade during IVD degeneration of aging.

### Example 2

PTH directly induces cAMP production and phosphorylation of CREB in NP cells.

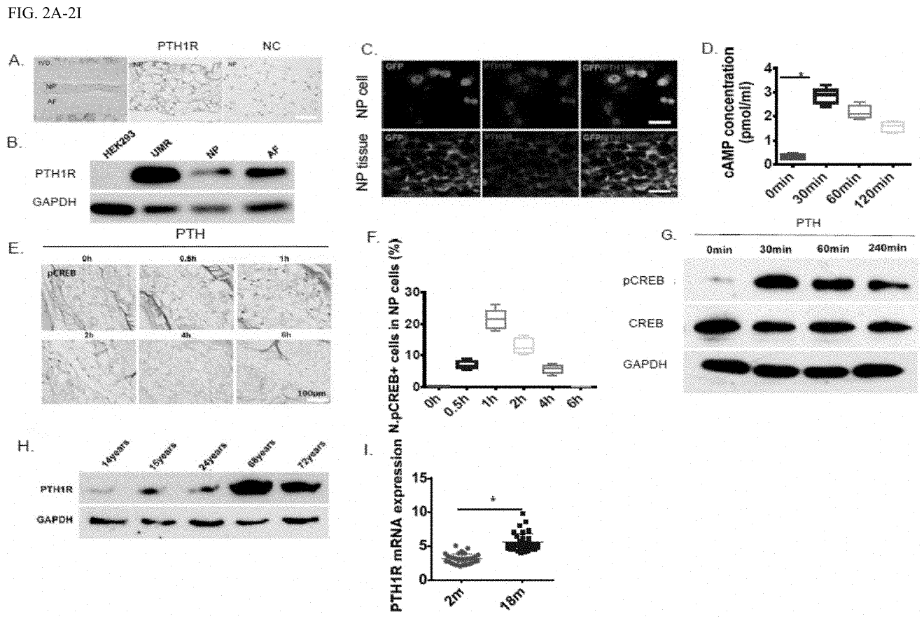

We examined whether PTH activates its downstream signaling directly in NP cells as PTH gland evolved in amphibians suggests its function for adaptation of vertebrates on land. Immunostaining of L3-L4 disc sections showed that PTH1R was expressed in NP cells (brown area) (FIG. 2A). Western blot analysis further demonstrated the expression of PTH1R in NP and AF (FIG. 2B). To validate that PTH1R expression in NP cells is of notochordal origin, we crossed ROSA26-GFP^{flox/flox} mice with Noto^{Cre} mice to generate mice with GFP fluorescence in the notochord cell lineage (NotoCre::ROSA26-GFP). GFP⁺ IVD cells isolated from the Noto^{Cre}::ROSA26-GFP mice co-localized with PTH1R (FIG. 2C, upper panel) and immunofluorescence staining of the IVD sections also demonstrated that PTH1R was colocalized with GFP⁺ cells in the NP area (FIG. 2C, lower panel), confirming that PTH1R is expressed in the notochord-derived NP cells.

To elucidate whether PTH stimulates downstream intracellular signaling, we measured the level of PTH-induced cyclic adenosine monophsphate (cAMP) production in NP cells. The cAMP level in NP cells peaked at 30 minutes with PTH (1-34) (100 nmol) treatment (FIG. 2D). Number of pCREB+ cells increased in NP area at 30 minutes and peaked at 1 hour after treatment (FIGS. 2E, F). Western blot analysis also demonstrated that PTH induced phosphorylation of CREB at 30 min in NP cells (FIG. 2G). Importantly, PTH1R protein levels in the human NP and AF cells were relatively low in young and adults but significantly increased during aging in Western blot analysis of patient lumbar IVD specimens (FIG. 2H). Similarly, the levels of PTH1R mRNA expression in NP tissue were relatively low in young adults and increased with aging. (FIG. 2I). Taken together, these results reveal PTH direct signaling in NP cells with potential function in maintenance of IVD during aging.

### Example 3

iPTH attenuates disc degeneration by inducing integrin αvβ6 expression in activation of TGF-α.

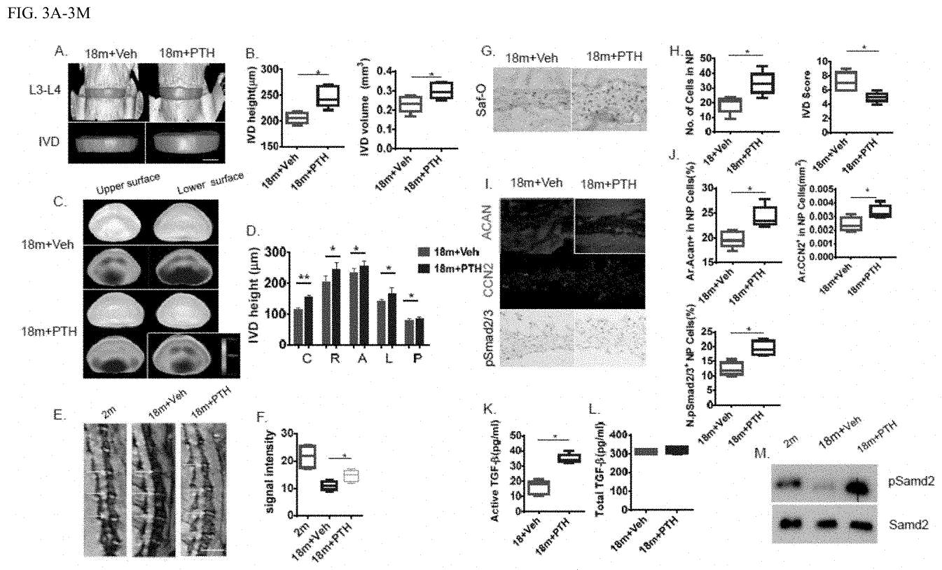

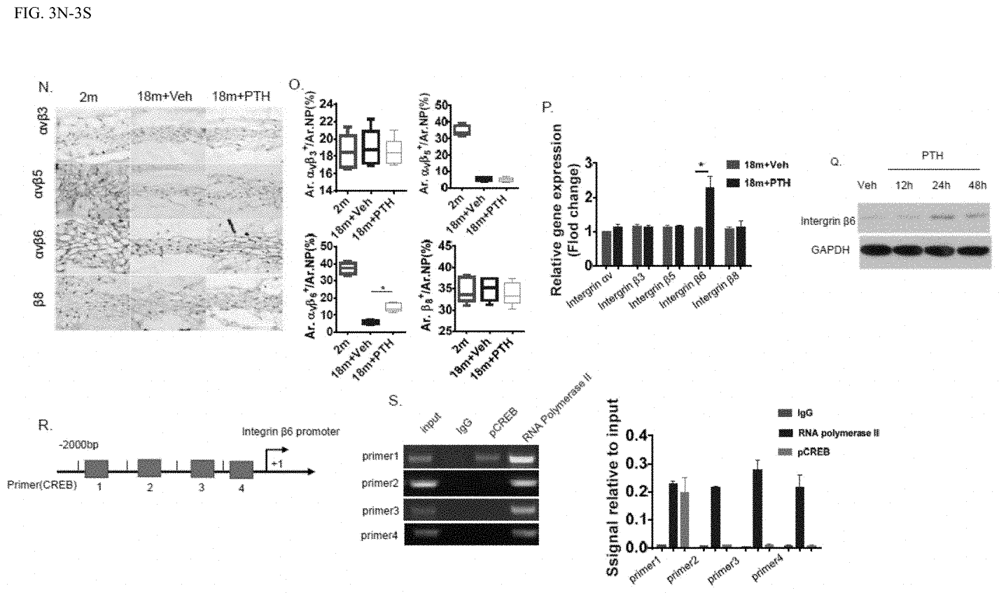

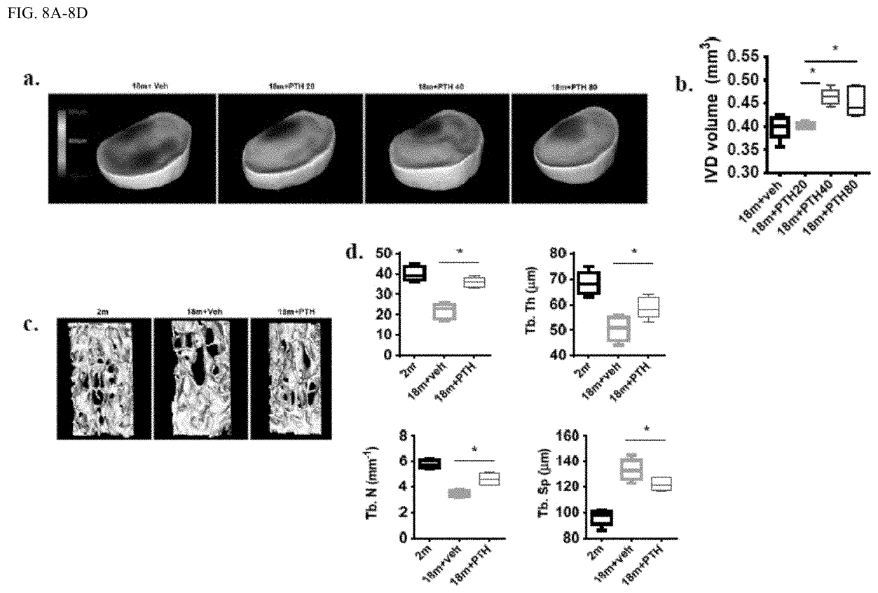

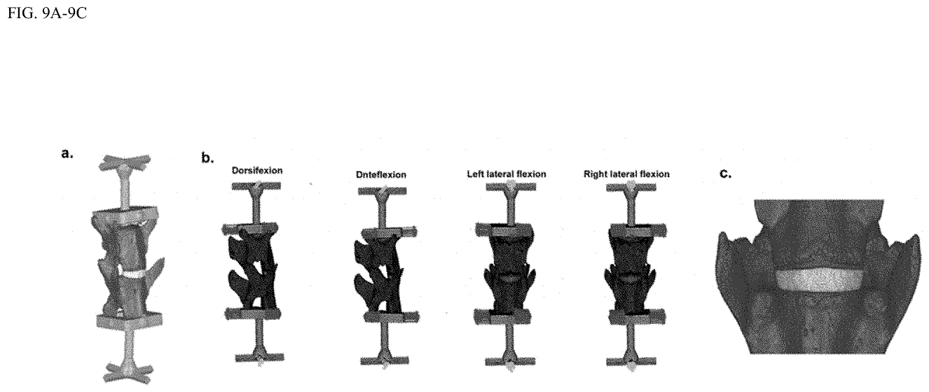

To examine the potential effect of intermittent PTH (iPTH) on IVD degeneration, we injected aged mice with PTH, a C-terminal truncated synthetic analogue of human PTH (1-34) daily for eight weeks with different doses. iPTH 40 μg/kg and 80 μg/kg significantly improved the IVD morphology and the dose of 40 μg/kg was chosen for the rest of the study (FIGS. 8A, B). Specifically, IVD height and volume were increased with iPTH in the 18-month-old mice relative to vehicle mice (FIGS. 3A, B), with increase of bone volume (BV/TV) and connectivity (Tb. Con) (FIGS. 8C, D). Moreover, significant increase in IVD thickness in 3D images in five different areas with iPTH, especially the central area of IVD (FIGS. 3C, D). The MRI signal intensity of lumbar IVDs significantly increased with iPTH injection (FIGS. 3E, F). The number of NP cells significantly increased with decrease of IVD score (FIGS. 3G, H) and the level of aggrecan and CCN2⁺ increased with iPTH (FIGS. 3I, J). Furthermore, the number of pSmad2/3⁺ NP cells increased with iPTH treatment (FIGS. 3I, J), indicating that PTH induces activation of TGF-β in IVD. Indeed, level of active TGF-β increased in IVDs with iPTH treatment while total TGF-β level was not changed in ELISA (FIGS. 3H, L). Western blot analysis confirmed that pSmad2 increased with iPTH (FIG. 3M).

To determine the mechanism by which PTH induces the activation of TGF-β in IVD, we examined whether PTH increased the expression of αvβ integrins, which mediate activation of latent TGF-β. IVD sections prepared from iPTH- or vehicle-treated 18-month-old mice were immunostained with individual antibodies against αvβ3, αvβ5, αvβ6 and (38, respectively and integrin levels compared to those of 2-month-old mice. The results showed that the expressions of αvβ5 and αvβ6 integrin were significantly decreased in the 18-month-old mice relative to 2-month-old mice, while the expression levels of αvβ3 and β8 remained relatively unchanged. Importantly, PTH only significantly increased the level of αvβ6 in 18-month old mice (FIGS. 3N, O). To validate the regulation of αvβ6 integrin by iPTH, we performed qRTPCR to quantify the effect of PTH treatment on individual integrin mRNA expression. Consistently, PTH specifically induced mRNA expression of β6 integrin in NP cells of the aged mice (FIG. 3P). PTH also significantly stimulated the protein level of β6 integrin in a time dependent manner (FIG. 3Q). Thus, PTH activates latent TGF-β by increasing αvβ6 expression in NP tissue, offering a potential therapeutic target for lumbar disc disease.

To determine the mechanism of PTH-induced β6 integrin gene transcription, we performed chromatin immunoprecipitation assay with 4 different potential pCREB binding sites (Primers 1, 2, 3 and 4) in the β6 integrin promoter. Immunoprecipitation results revealed that pCREB specifically binds to the most distal CREB site in the β6-integrin promoter (Primer 1) (FIGS. 3R, S). Taken together, these results demonstrate that PTH activates expression of β6 integrin by inducing direct binding of pCREB to the β6 integrin promoter in NP cells.

### Example 4

Conditional Knockout of PTH1R in NP cells accelerates disc degeneration during aging.

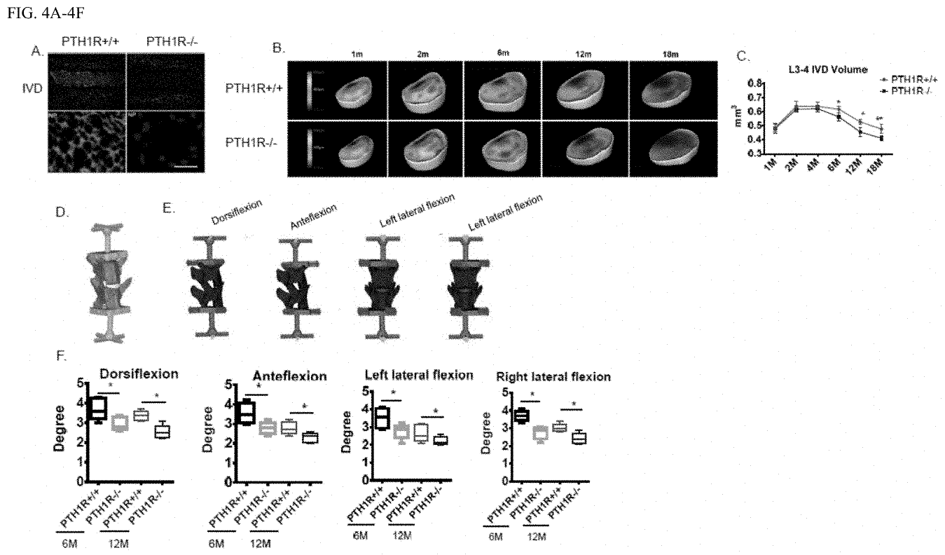

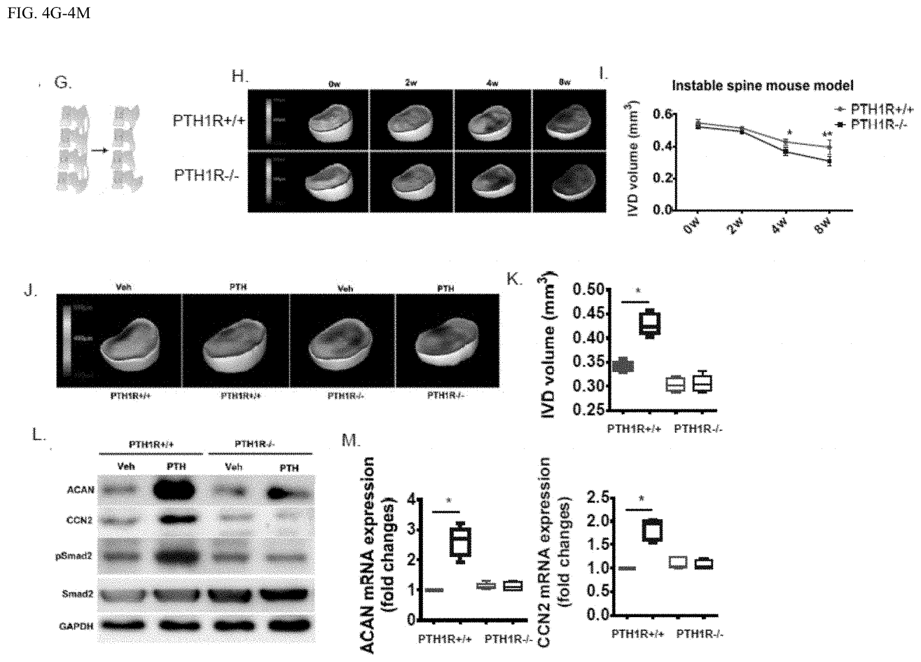

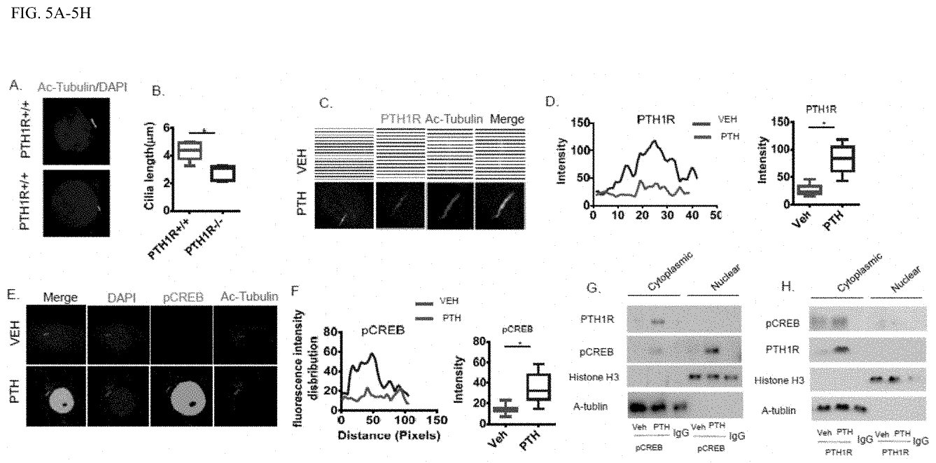

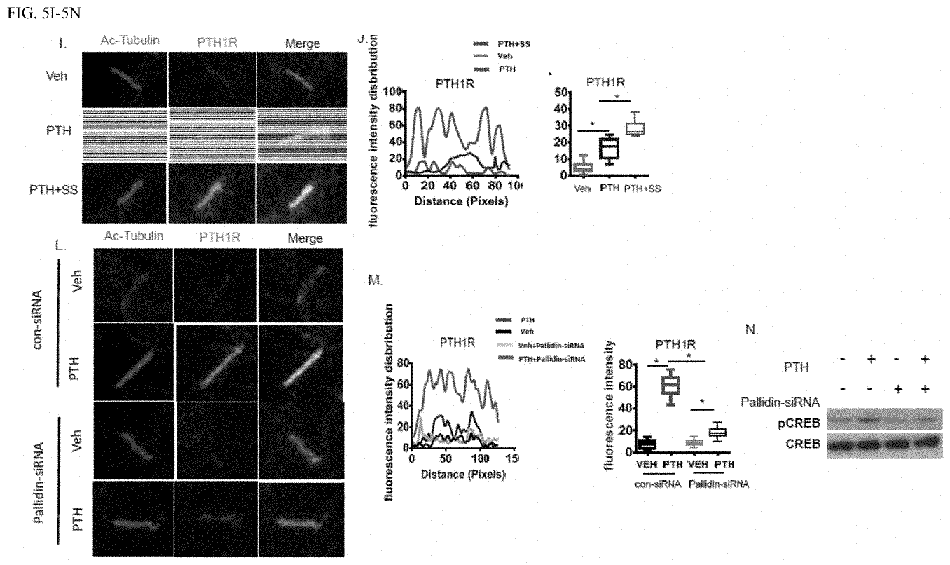

To investigate the role of PTH signaling in NP cells during aging, we crossed Noto^{Cre} mice with PTH1R^{flox/flox} mice to delete PTH1R gene specifically in notochord-derived NP cells (Noto^{Cre}::PTH1R^{flox/flox}, named “PTH1R KO mice” thereafter). PTH1R expression was undetectable in the NP cells in disc sections of PTH1R KO mice (FIG. 4A). The IVD changes were evaluated with PPCT in PTH1R KO mice and their wild-type littermates at different ages. We found that IVD volumes decreased starting at 6-months of age relative to their aged matched wild type littermates (named “PTH1R^{+/+} mice” thereafter), whereas IVD volumes at postnatal and adulthood remained normal in PTH1R KO mice (FIGS. 4B, C), suggesting that a critical role of PTH in maintaining function of IVD during aging. In human, higher levels of PTH1R expression were also observed only in aged IVDs (FIG. 2I). To assess IVD function, the 3D finite element analysis model based on PPCT image acquisition were used for IVD flexibility testing, including dorsiflexion, ante-flexion, left and right lateral flexion (FIGS. 4D, E). Flexibility of IVDs in all directions showed significantly progressive decrease in PTH1R KO mice starting at 6 months (FIG. 4F). Moreover, we generated a spine destabilized mouse model by resection of the intervertebral ligament of adjoining lumbar vertebrae to assess whether PTH regulates the function of discs under unstable mechanical loading environment (FIG. 4G). IVD volume was decreased in 2-month-old PTH1R^{-/-} mice 4 weeks post-surgery, which was much earlier than that in the stable model where significant decrease started at 6-months of age and is much pronounced at 12-months (FIGS. 4H, I).

We then examined whether deletion of PTH1R in NP cells could affect iPTH anabolic effect on IVD. Increase of IVD volumes by iPTH in 12-month-old PTH1R^{+/+} mice was abolished in PTH1R KO mice (FIGS. 4J, K). Furthermore, Western blot analysis showed that the PTH induced increase in levels of pSmad2, ACAN and CCN2 seen in NP tissue was diminished in the PTH1R KO mice (FIG. 4L). PTH significantly increased the mRNA expression of ACAN and CCN2 in NP cells of wild-type mice by qRT-PCR, which were blunted in PTH1R KO mice (FIG. 4M). Thus, PTH signaling in NP cells is essential for disc homeostasis, especially during aging.

### Example 5

PTH stimulates transport of PTH1R to cilia of NP cells.

To understand if mechanical stress regulates PTH signaling in NP cells, we investigated whether primary cilia in the NP cells regulates PTH signaling as PTH1R is a GPCR and found in primary cilia. Immunostaining of acetylated tubulin demonstrated that primary cilia were present in the NP cells of wild-type mice and the length of primary cilia significantly decreased in PTH1R KO mice (FIGS. 5A, B). Importantly, PTH stimulated translocation of PTH1R to cilia significantly, starting in 10 minutes (FIGS. 5C, D). Co-immunostaining of pCREB with acetylated tubulin also revealed that pCREB increased in cilia (FIGS. 5E, F), suggesting cilia in regulation of PTH signaling. To examine whether PTH1R and pCREB formed a complex, co-immunoprecipitation with antibody against pCREB and blotted with PTH1R demonstrated that the interaction between PTH1R and pCREB was only in the cytoplasmic acetyl tubulin abstracts including cilia (FIG. 5G). Similarly, coimmunoprecipitation with antibody against PTH1R showed the interaction only present in the cytoplasmic acetyl tubulin abstracts (FIG. 5H).

We then examined whether mechanical stress regulates PTH signaling through cilia. NP cells were applied with sheer stress, and translocation of PTH1R to cilia was significantly enhanced by sheer stress (FIGS. 5I, J). Pallidin mediates transport of proteins to cilia. We therefore knocked down pallidin with siRNA to validate PTH-enhanced translocation of PTH1R to cilia. The knockdown of pallidin blocked PTH-enhanced transport of PTH1R to cilia and phosphorylation of CREB (FIGS. 5K-M). Taken together, PTH stimulates transport of PTH1R to primary cilia and sheer stress enhances the transport.

### Example 6

Disruption of cilia decreases IVD volume, PTH signaling and TGF-β activity in NP cells.

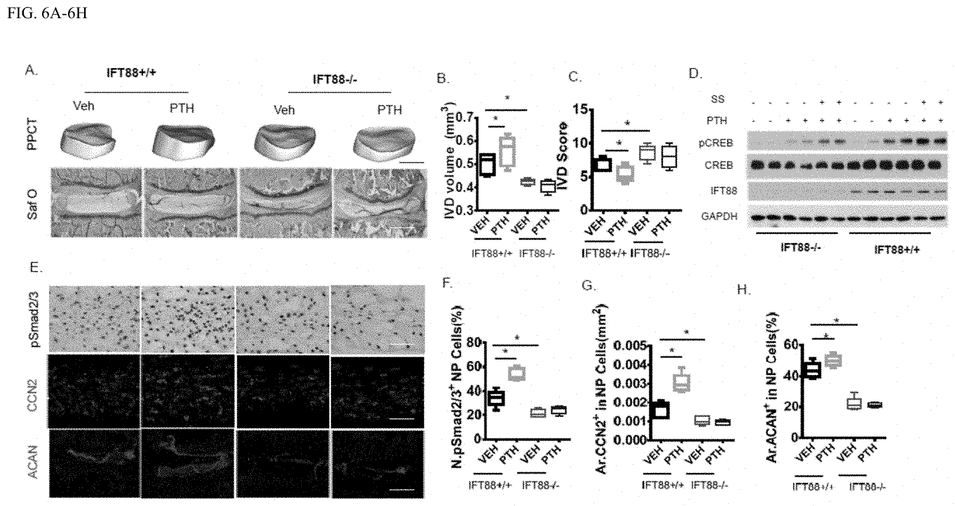

To examine the role of cilia in maintaining IVD function, we crossed IFT88^{flox/flox} mice with Noto^{Cre/+} mice to generate IFT88::Noto-Cre mice (IFT88^{-/-}) with disruption of cilia specifically in NP cells. The IVD volume was significantly decreased with disruption of cilia specifically in NP cells in IFT88^{-/-} mice relative to their wild-type mice (FIGS. 6A, B), and the IVD scores were increased consequently (FIGS. 6A, C). Furthermore, iPTH-increased IVD volume was blunted in IFT88^{-/-} mice relative to their wild-type mice (FIGS. 6A, B) and the improvement of IVD scores by iPTH was also impaired (FIGS. 6A, C). To validate that mechanical stress regulates PTH signaling through transport of PTH1R to cilia, we examined whether PTH stimulates CREB phosphorylation in NP cells with disruption of primary cilia. Western blot analysis showed that PTH significantly stimulated the phosphorylation of CREB and sheer stress enhanced the phosphorylation in wild-type mice (FIG. 6D), however, disruption of primary cilia significantly reduced levels of CREB phosphorylation by either PTH or sheer stress in IFT88^{-/-} NP cells. Importantly, immunohistochemical staining demonstrated that PTH significantly stimulated the number of pSmad2/3⁺ NP cells and the levels of CCN2 and aggrecan in wild-type mice relative to vehicle mice and the effect was blunted by disruption of cilia specifically in NP cells in IFT88^{-/-} mice (FIGS. 6E-H). These data demonstrate that mechanical stress enhances PTH signaling in NP cells via primary cilia to maintain IVD function.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. Boos, N., et al. Classification of age—related changes in lumbar intervertebral discs: 2002 Volvo Award in basic science. Spine 27, 2631-2644 (2002).
2. Miller, J. A., Schmatz, C. & Schultz, A. B. Lumbar disc degeneration: correlation with age, sex, and spine level in 600 autopsy specimens. Spine 13, 173-178 (1988).
3. Raj, P. P. Intervertebral disc: anatomy—physiology—pathophysiology—treatment. Pain practice: the official journal of World Institute of Pain 8, 18-44 (2008).
4. Vos, T., et al. Years lived with disability (YLDs) for 1160 sequelae of 289 diseases and injuries 1990-2010: a systematic analysis for the Global Burden of Disease Study 2010. Lancet 380, 2163-2196 (2012).
5. Waddell, G. Low back pain: a twentieth century health care enigma. Spine 21, 2820-2825 (1996).
6. Frymoyer, J. W. Back pain and sciatica. The New England journal of medicine 318, 291-300 (1988).
7. Trout, J. J., Buckwalter, J. A. & Moore, K. C. Ultrastructure of the human intervertebral disc: II. Cells of the nucleus pulposus. The Anatomical record 204, 307-314 (1982).
8. McCann, M. R., Tamplin, O. J., Rossant, J. & Seguin, C. A. Tracing notochord—derived cells using a Notocre mouse: implications for intervertebral disc development. Dis Model Mech 5, 73-82 (2012).
9. Schmidt, M. B., Mow, V. C., Chun, L. E. & Eyre, D. R. Effects of Proteoglycan Extraction on the Tensile Behavior of Articular—Cartilage. J Orthopaed Res 8, 353-363 (1990).
10. Aigner, T., Bertling, W., Stoss, H., Weseloh, G. & Vondermark, K. Independent Expression of Fibril—Forming Collagen—I, Collagen—Ii, and Collagen—Iii in Chondrocytes of Human Osteoarthritic Cartilage. J Clin Invest 91, 829-837 (1993).
11. Nishimura, G., et al. Identification of COL2A1 mutations in platyspondylic skeletal dysplasia, Torrance type. J Med Genet 41, 75-79 (2004).
12. Zankl, A., et al. Dominant negative mutations in the C-propeptide of COL2A1 cause platyspondylic lethal skeletal dysplasia, torrance type, and define a novel subfamily within the type 2 collagenopathies. Am J Med Genet A 133A, 61-67 (2005).
13. Erwin, W. M., Ashman, K., O'Donnel, P. & Inman, R. D. Nucleus pulposus notochord cells secrete connective tissue growth factor and up—regulate proteoglycan expression by intervertebral disc chondrocytes. Arthritis Rheum 54, 3859-3867 (2006).
14. Yang, H. L., et al. TGF-beta 1 Suppresses Inflammation in Cell Therapy for Intervertebral Disc Degeneration. Sci Rep—Uk 5 (2015).
15. Pattison, S. T., Melrose, J., Ghosh, P. & Taylor, T. K. F. Regulation of gelatinase—A (MMP-2) production by ovine intervertebral disc nucleus pulposus cells grown in alginate bead culture by transforming growth factor-beta(1) and insulin like growth factor—I. Cell Biol Int 25, 679-689 (2001).
16. Tran, C. M., et al. Regulation of CCN2/Connective Tissue Growth Factor Expression in the Nucleus Pulposus of the Intervertebral Disc Role of Smad and Activator Protein 1 Signaling. Arthritis Rheum 62, 1983-1992 (2010).
17. Eguchi, T., et al. Novel transcription factor—like function of human matrix metalloproteinase 3 regulating the CTGF/CCN2 gene. Mol Cell Biol 28, 2391-2413 (2008).
18. Yuan, X., Serra, R. A. & Yang, S. Y. Function and regulation of primary cilia and intraflagellar transport proteins in the skeleton. Ann Ny Acad Sci 1335, 78-99 (2015).
19. Green, J. A., et al. Recruitment of beta—Arrestin into Neuronal Cilia Modulates Somatostatin Receptor Subtype 3 Ciliary Localization. Mol Cell Biol 36, 223-235 (2016).
20. Leaf, A. & Von Zastrow, M. Dopamine receptors reveal an essential role of IFT-B, KIF17, and Rab23 in delivering specific receptors to primary cilia. Elife 4 (2015).
21. Liem, K. F., et al. The IFT-A complex regulates Shh signaling through cilia structure and membrane protein trafficking. Journal of Cell Biology 197, 789-800 (2012).
22. Kopinke, D., Roberson, E. C. & Reiter, J. F. Ciliary Hedgehog Signaling Restricts Injury-Induced Adipogenesis. Cell 170, 340-+(2017).
23. McIntyre, J. C., Hege, M. M. & Berbari, N. F. Trafficking of ciliary G protein-coupled receptors. Method Cell Biol 132, 35-54 (2016).
24. Schou, K. B., Pedersen, L. B. & Christensen, S. T. Ins and outs of GPCR signaling in primary cilia. Embo Rep 16, 1099-1113 (2015).
25. Oliazadeh, N., Gorman, K. F., Eveleigh, R., Bourque, G. & Moreau, A. Identification of Elongated Primary Cilia with Impaired Mechanotransduction in Idiopathic Scoliosis Patients. Sci Rep-Uk 7 (2017).
26. Grimes, D. T., et al. Zebrafish models of idiopathic scoliosis link cerebrospinal fluid flow defects to spine curvature. Science 352, 1341-1344 (2016).
27. Okabe, M. & Graham, A. The origin of the parathyroid gland. Proceedings of the National Academy of Sciences of the United States of America 101, 17716-17719 (2004).
28. Kawane, T., Mimura, J., Yanagawa, T., Fujii-Kuriyama, Y. & Horiuchi, N. Parathyroid hormone (PTH) down-regulates PTH/PTH-related protein receptor gene expression in UMR-106 osteoblast-like cells via a 3′,5′-cyclic adenosine monophosphate-dependent, protein kinase A—independent pathway. J Endocrinol 178, 247-256 (2003).
29. Swarthout, J. T., D'Alonzo, R. C., Selvamurugan, N. & Partridge, N.C. Parathyroid hormone—dependent signaling pathways regulating genes in bone cells. Gene 282, 1-17 (2002).
30. Qiu, T., et al. TGF—beta type II receptor phosphorylates PTH receptor to integrate bone remodeling signalling. Nat Cell Biol 12, 224-U229 (2010).
31. Wan, M., et al. Parathyroid hormone signaling through low—density lipoprotein—related protein 6. Gene Dev 22, 2968-2979 (2008).
32. Canalis, E., Centrella, M., Burch, W. & Mccarthy, T. L. Insulin—Like Growth Factor—I Mediates Selective Anabolic Effects of Parathyroid—Hormone in Bone Cultures. J Clin Invest 83, 60-65 (1989).
33. Pfeilschifter, J., et al. Parathyroid—Hormone Increases the Concentration of Insulin-Like Growth-Factor-I and Transforming Growth-Factor-Beta-1 in Rat Bone. J Clin Invest 96, 767-774 (1995).
34. Yu, B., et al. Parathyroid hormone induces differentiation of mesenchymal stromal/stem cells by enhancing bone morphogenetic protein signaling. J Bone Miner Res 27, 2001-2014 (2012).
35. Prisby, R., et al. Intermittent PTH(1-84) Is Osteoanabolic but Not Osteoangiogenic and Relocates Bone Marrow Blood Vessels Closer to Bone—Forming Sites. J Bone Miner Res 26, 2583-2596 (2011).
36. Munger, J. S. & Sheppard, D. Cross talk among TGF—beta signaling pathways, integrins, and the extracellular matrix. Cold Spring Harbor perspectives in biology 3, a005017 (2011).
37. Nishimura, S. L. Integrin—mediated transforming growth factor—beta activation, a potential therapeutic target in fibrogenic disorders. The American journal of pathology 175, 1362-1370 (2009).
38. Mamuya, F. A. & Duncan, M. K. aV integrins and TGF—beta—induced EMT: a circle of regulation. Journal of cellular and molecular medicine 16, 445-455 (2012).
39. Weiss, R. E. & Watabe, N. Studies on the biology of fish bone. III. Ultrastructure of osteogenesis and resorption in osteocytic (cellular) and anosteocytic (acellular) bones. Calcified tissue international 28, 43-56 (1979).
40. Glowacki, J., Cox, K. A., O'Sullivan, J., Wilkie, D. & Deftos, L. J. Osteoclasts can be induced in fish having an acellular bony skeleton. Proceedings of the National Academy of Sciences of the United States of America 83, 4104-4107 (1986).
41. Witten, P. E. & Huysseune, A. A comparative view on mechanisms and functions of skeletal remodelling in teleost fish, with special emphasis on osteoclasts and their function. Biological reviews of the Cambridge Philosophical Society 84, 315-346 (2009).
42. Moss, M. L. Studies of the Acellular Bone of Teleost Fish. V. Histology and Mineral Homeostasis of Fresh-Water Species. Acta anatomica 60, 262-276 (1965).
43. Kurland, E. S., et al. Parathyroid hormone as a therapy for idiopathic osteoporosis in men: effects on bone mineral density and bone markers. The Journal of clinical endocrinology and metabolism 85, 3069-3076 (2000).
44. Morley, P., Whitfield, J. F. & Willick, G. E. Parathyroid hormone: an anabolic treatment for osteoporosis. Current pharmaceutical design 7, 671-687 (2001).
45. Canalis, E., Giustina, A. & Bilezikian, J. P. Mechanisms of anabolic therapies for osteoporosis. The New England journal of medicine 357, 905-916 (2007).
46. Finkelstein, J. S., et al. The effects of parathyroid hormone, alendronate, or both in men with osteoporosis. The New England journal of medicine 349, 1216-1226 (2003).
47. Sapp, G. Evolution: The First Four Billion Years. Libr J 133, 156-156 (2008).
48. Madiraju, P., Gawri, R., Wang, H., Antoniou, J. & Mwale, F. Mechanism of parathyroid hormonemediated suppression of calcification markers in human intervertebral disc cells. European cells & materials 25, 268-283 (2013).
49. Zhou, Z., et al. Enhancement of Lumbar Fusion and Alleviation of Adjacent Segment Disc Degeneration by Intermittent PTH(1-34) in Ovariectomized Rats. J Bone Miner Res 31, 828-838 (2016).
50. Risbud, M. V., Schaer, T. P. & Shapiro, I. M. Toward an understanding of the role of notochordal cells in the adult intervertebral disc: from discord to accord. Developmental dynamics: an official publication of the American Association of Anatomists 239, 2141-2148 (2010).
51. Sivakamasundari, V. & Lufkin, T. Bridging the Gap: Understanding Embryonic Intervertebral Disc Development. Cell & developmental biology 1 (2012).
52. Gower, W. E. & Pedrini, V. Age-related variations in proteinpolysaccharides from human nucleus pulposus, annulus fibrosus, and costal cartilage. The Journal of bone and joint surgery. American volume 51, 1154-1162 (1969).
53. Antoniou, J., et al. The human lumbar intervertebral disc: evidence for changes in the biosynthesis and denaturation of the extracellular matrix with growth, maturation, ageing, and degeneration. J Clin Invest 98, 996-1003 (1996).
54. Tran, C. M., Shapiro, I. M. & Risbud, M. V. Molecular regulation of CCN2 in the intervertebral disc: lessons learned from other connective tissues. Matrix biology: journal of the International Society for Matrix Biology 32, 298-306 (2013).
55. Serra, R., et al. Expression of a truncated, kinase-defective TGF-beta type II receptor in mouse skeletal tissue promotes terminal chondrocyte differentiation and osteoarthritis. The Journal of cell biology 139, 541-552 (1997).
56. Yang, X., et al. TGF-beta/Smad3 signals repress chondrocyte hypertrophic differentiation and are required for maintaining articular cartilage. The Journal of cell biology 153, 35-46 (2001).
57. Bian, Q., et al. Mechanosignaling activation of TGF beta maintains intervertebral disc homeostasis. Bone Res 5 (2017).
58. Cao, Y., et al. 3D visualization of the lumbar facet joint after degeneration using propagation phase contrast micro-tomography. Sci Rep 6, 21838 (2016).
59. Momose, A., Takeda, T., Itai, Y. & Hirano, K. Phase-contrast X-ray computed tomography for observing biological soft tissues. Nature medicine 2, 473-475 (1996).
60. Zhou, S. A. & Brahme, A. Development of phase-contrast X-ray imaging techniques and potential medical applications. Physica medica: PM: an international journal devoted to the applications of physics to medicine and biology: official journal of the Italian Association of Biomedical Physics 24, 129-148 (2008).
61. Wilkins, S. W., Gureyev, T. E., Gao, D., Pogany, A. & Stevenson, A. W. Phase-contrast imaging using polychromatic hard X-rays. Nature 384, 335-338 (1996).
62. Eggl, E., et al. X-ray phase-contrast tomography with a compact laser-driven synchrotron source. Proceedings of the National Academy of Sciences of the United States of America 112, 5567-5572 (2015).
63. Kobayashi, T., et al. PTHrP and Indian hedgehog control differentiation of growth plate chondrocytes at multiple steps. Development 129, 2977-2986 (2002).
64. Bedore, J., et al. Impaired Intervertebral Disc Development and Premature Disc Degeneration in Mice With Notochord-Specific Deletion of CCN2. Arthritis Rheum 65, 2634-2644 (2013).
65. Ben Abdelkhalek, H., et al. The mouse homeobox gene Not is required for caudal notochord development and affected 1725 by the truncate mutation. Gene Dev 18, 1725-1736 (2004).
66. Sakai, D., et al. Exhaustion of nucleus pulposus progenitor cells with ageing and degeneration of the intervertebral disc. Nat Commun 3 (2012).
67. Masuda, K., et al. A novel rabbit model of mild, reproducible disc degeneration by an anulus needle puncture: Correlation between the degree of disc injury and radiological and histological appearances of disc degeneration. Spine 30, 5-14 (2005).
68. Kim, H. J., et al. The influence of facet joint orientation and tropism on the stress at the adjacent segment after lumbar fusion surgery: a biomechanical analysis. Spine Journal 15, 1841-1847 (2015).
69. Malandrino, A., et al. The role of endplate poromechanical properties on the nutrient availability in the intervertebral disc. Osteoarthr Cartilage 22, 1053-1060 (2014).
70. Nyman, J. S., et al. Predicting mouse vertebra strength with micro-computed tomography-derived finite element analysis. Bonekey Rep 4 (2015).

1. A method comprising administration to a subject a composition comprising parathyroid hormone (PTH) or a functional fragment or analog thereof, wherein the administration results in one or more of:
regeneration of aged intravertebral discs (IVDs) in the subject,
increasing total tissue volume of the nucleus pulposus (NP) and/or anulus fibrosus (AF) and/or cartilaginous endplates (EP) in the IVDs of the subject,
reducing NP cell apoptosis in the IVDs of the subject, and
increasing the levels of active TGF-b in the NP cells of the IVDs of the subject.

2. The method of claim 1, wherein the subject is a human subject.

3. The method of claim 1, wherein the subject is a human subject suffering from or at risk of suffering from IVD degeneration and/or lumbar disc disease (LDD).

4. The method of claim 1, wherein the PTH or a functional fragment or analog thereof is administered with a pharmaceutically acceptable carrier.

5. The method of claim 1, wherein the PTH or a functional fragment or analog thereof is selected from the group consisting of PTH(1-28), PTH(1-31), PTH(1-34), PTH(1-37), PTH(1-38), PTH(1-41), and recombinant rPTH(1-34).

6. The method of claim 1, wherein the PTH or a functional fragment or analog thereof is administered to the subject at a dose of between 10 mg/kg/day to 500 mg/kg/day.

7. The method of claim 1, wherein the PTH or a functional fragment or analog thereof is administered intramuscularly, intradermally, or intraperitoneally.
